SEQUENCE LISTING (15 sequences)

SEQ ID NO: 1 - protein, 17 aa, Artificial Sequence
  17-amino acid peptide
KKFRRLKWKYKGKFWFW

SEQ ID NO: 2 - protein, 17 aa, Artificial Sequence
  17-amino acid peptide
KKYRRFRWKFKGKFWFW

SEQ ID NO: 3 - protein, 17 aa, Artificial Sequence
  17-amino acid peptide
RRYKKFKWRYRGRFWFW

SEQ ID NO: 4 - protein, 18 aa, Artificial Sequence
  18-amino acid peptide
GRRYRRFWKFRGKWFFWG

SEQ ID NO: 5 - protein, 19 aa, Artificial Sequence
  19-amino acid peptide
GKKYRRFRWKFKGKWFWFG

SEQ ID NO: 6 - protein, 19 aa, Artificial Sequence
  19-amino acid peptide
GRRYRRFRWRFRGRFWFWG

SEQ ID NO: 7 - protein, 19 aa, Artificial Sequence
  19-amino acid peptide
GRRYKKFRWKFKGRWFWFG

SEQ ID NO: 8 - protein, 19 aa, Artificial Sequence
  19-amino acid peptide
GRRYRRFRWRFRGRFWFWG

SEQ ID NO: 9 - protein, 19 aa, Artificial Sequence
  19-amino acid peptide, Pep19-4LF
GKKYRRFRWKFKGKLFLFG

SEQ ID NO: 10 - protein, 20 aa, Artificial Sequence
  20-amino acid peptide, Pep19-2.5
GCKKYRRFRWKFKGKFWFWG

SEQ ID NO: 11 - protein, 21 aa, Artificial Sequence
  21-amino acid peptide
GCKKFRRFKLKCKQKLWLWCG

SEQ ID NO: 12 - protein, 21 aa, Artificial Sequence
  21-amino acid peptide
GCKKFRRLKWKYKGKFWFWCG

SEQ ID NO: 13 - protein, 21 aa, Artificial Sequence
  21-amino acid peptide
GCRRYRRFRWKFRGRFWFWCG

SEQ ID NO: 14 - protein, 21 aa, Artificial Sequence
  21-amino acid peptide
GCRRYKKFKWRYRGRFWFWCG

SEQ ID NO: 15 - protein, 21 aa, Artificial Sequence
  21-amino acid peptide
GCRRFRRFRWRYRGRFWFWCG

(19) United States
(12) Patent Application Publication
(10) Pub. No.: US 2019/0092814 A1
(43) Pub. Date: Mar. 28, 2019

(54) Peptide

(71) Applicant: Queen Mary University of London, London (GB)

(72) Inventors: Christoph Thiemermann, London (GB); Lukas Martin, London (GB); Tobias S. Schuerholz, Rostock (DE); Klaus Brandenburg, Borstel (DE)

(21) Appl. No.: 16/136,334

(22) Filed: Sep. 20, 2018

(30) Foreign Application Priority Data
Sep. 22, 2017 (GB) 1715379.2

(51) Int. Cl. C07K 7/08; A61P 7/04; A61K 9/00

(52) U.S. Cl. CPC C07K 7/08; A61K 38/10; A61K 9/0019; A61P 7/04

(57) ABSTRACT

The present invention relates to a method of preventing or treating hemorrhagic shock (HS), or ameliorating symptoms associated with HS in an individual, by administering an effective amount of a linear, non-cyclic peptide comprising 17 to 21 amino acids, wherein the amino acids in positions (1) to (21) of the peptide, counted from the N-terminus, are as follows: (1) G, S or lacking; (2) C or lacking; (3) K or R; (4) K or R; (5) Y or F; (6) K or R; (7) K or R; (8) F, W or L; (9) K, R or lacking; (10) W, L or F; (11) K or R; (12) F, Y or C; (13) K or R; (14) G or Q; (15) K or R; (16) F, L or W; (17) F or W; (18) F, L or W; (19) W or F; (20) C or lacking; (21) G or lacking, wherein when position (9) is lacking, positions (1) and (21) cannot be lacking.

### FIELD OF THE INVENTION

The present invention relates to a method of preventing or treating hemorrhagic shock (HS), or ameliorating symptoms associated with HS in an individual using a linear, non-cyclic peptide comprising 17 to 21 amino acids.

### BACKGROUND TO THE INVENTION

Severe injuries account for 9% of the deaths worldwide. Although guidelines for the early management of HS, including resuscitation and organ support strategies, have decreased the rates of immediate (on scene/within 60 min) and early (emergency department and operating room/within 1-4 h) deaths, post-injury multiple organ failure (MOF) is still associated with significant morbidity and mortality.

Therapeutic agents that reduce the incidence and severity of MOF following HS could, therefore, have a major global impact on patient outcomes and resource utilization. The MOF after HS is associated with excessive systemic inflammation, secondary to the release of damage-associated molecular patterns (DAMPs) from extensive tissue damage and ischemia reperfusion injury. To date, there are no specific pharmacological interventions used clinically to prevent MOF following/associated with HS. Thus, there is a need for better management or treatment of HS and alleviation of its associated symptoms.

### SUMMARY OF THE INVENTION

The present inventors have identified a method of treating HS using antimicrobial peptides with a primary structure (amino acid sequence) based on the primary structure of an animal LPS-binding protein peptide from Limulus polyphemus, the Limulus anti-LPS factor (LALF).

The invention provides a method of preventing or treating hemorrhagic shock (HS), or ameliorating symptoms associated with HS in an individual, wherein the method comprises administering to the individual an effective amount of a peptide or a variant thereof, wherein the peptide or variant thereof is linear and non-cyclic, and comprises 17 to 21 amino acids, and wherein the amino acids in positions (1) to (21), counted from the N-terminus, are as follows:
(1) G, S or lacking;
(2) C or lacking;
(3) K or R;
(4) K or R;
(5) Y or F;
(6) K or R;
(7) K or R;
(8) F, W or L;
(9) K, R or lacking;
(10) W, L or F;
(11) K or R;
(12) F, Y or C;
(13) K or R;
(14) G or Q;
(15) K or R;
(16) F, L or W;
(17) F or W;
(18) F, L or W;
(19) W or F;
(20) C or lacking;
(21) G or lacking;
wherein when position (9) is lacking, positions (1) and (21) cannot be lacking,
and wherein the variant has up to one, two or three substitutions.

The invention also provides a peptide or a variant thereof for use in a method of preventing or treating hemorrhagic shock (HS), or ameliorating symptoms associated with HS in an individual, wherein the method comprises administering to the individual an effective amount of a peptide or variant thereof, wherein the peptide or variant thereof is linear and non-cyclic, and comprises 17 to 21 amino acids, and wherein the amino acids in positions (1) to (21), counted from the N-terminus, are as follows:
(1) G, S or lacking;
(2) C or lacking;
(3) K or R;
(4) K or R;
(5) Y or F;
(6) K or R;
(7) K or R;
(8) F, W or L;
(9) K, R or lacking;
(10) W, L or F;
(11) K or R;
(12) F, Y or C;
(13) K or R;
(14) G or Q;
(15) K or R;
(16) F, L or W;
(17) F or W;
(18) F, L or W;
(19) W or F;
(20) C or lacking;
(21) G or lacking;
wherein when position (9) is lacking, positions (1) and (21) cannot be lacking;
and wherein the variant has up to one, two or three substitutions.

### BRIEF DESCRIPTION OF THE FIGURES

FIG. 1: (A) LL-37 plasma levels in human healthy volunteers and trauma patients. Plasma concentrations of the cathelicidin LL-37 were assessed in control healthy volunteers (n=10) and in trauma patients (n=24) as well as in trauma hemorrhagic patients (n=23). Data are expressed as box and whisker min to max for n number of observations. +=mean value. *P<0.05 vs. healthy; ^{§}P<0.05 vs. trauma (Kruskall-Wallis test with Dunn's multiple comparisons test).

FIG. 2: Associations between the serum levels of LL-37 and clinical characteristics. Plasma concentrations of the cathelicidin LL-37 were assessed in control healthy volunteers (n=10) and in trauma patients (n=24) as well as in trauma hemorrhagic patients (n=23). Spearman rank-order correlation was applied to (A) admission systolic blood pressure, (B) admission heart rate, (C) admission lactate, (D) admission base deficit. Sp.r, Spearman rank (Sp.r) correlation coefficient r, CI, 95% confidence interval.

FIG. 3: LL-37 plasma levels in human healthy volunteers and a subgroup of trauma patients (ISS>24). Plasma concentrations of the cathelicidin LL-37 were assessed in control healthy volunteers (n=10) and in subgroup of trauma patients (n=11) and trauma hemorrhagic patients (n=21) with an Injury Severity Score (ISS) threshold of >24. Data are expressed as box and whisker min to max for n number of observations. +=mean value. *P<0.05 vs. healthy; § P<0.05 vs. trauma (Kruskall-Wallis test with Dunn's multiple comparisons test).

FIG. 4: Pep19-4LF or Pep19-2.5 attenuate the decline in MAP during resuscitation after HS. (A) HS-rats received continuous administration of low-dose (LD; 66 μg/kg×h) or high-dose (HD; 333 μg/kg×h) of Pep19-4LF or saline (vehicle) throughout 4 h after resuscitation. Sham animals were used as control and received saline or high-dose of Pep19-4LF. The MAP was recorded during the whole experiment. The following groups were studied: sham+vehicle (n=11); sham+Pep19-4LF-HD (n=6); HS+vehicle (n=12); HS+Pep19-4LF-LD (n=4); HS+Pep19-4LF-HD (n=8). (B) HS-rats received continuous administration of high-dose (HD; 333 μg/kg×h) of Pep19-4LF or Pep19-2.5 or saline (vehicle) throughout 4 h after resuscitation. Sham animals were used as control and received saline. The MAP was recorded during the whole experiment. The following groups were studied: sham+vehicle (n=11); HS+vehicle (n=12); HS+Pep19-4LF-HD (n=8); HS+Pep19-2.5-HD (n=8). In both (A) and (B), data are expressed as mean±SEM for n number of observations. Statistical analysis was performed using 2-way ANOVA followed by Bonferroni post hoc test. *P<0.05 vs HS+vehicle. ANOVA indicated analysis of variance; SEM, standard error of the mean.

FIG. 5: Pep19-4LF attenuates the organ injury and dysfunction caused by HS. (A) Serum urea, (B) serum creatinine, (C) creatinine clearance (CCr), (D) serum lactate, (E) serum alanine transaminase (ALT), (F) serum aspartate transaminase (AST), (G) serum amylase, and (H) serum lipase of HS or sham-operated rats. All parameters were assessed 4 h subsequent to HS. HS-rats received continuous administration of low-dose (LD; 66 μg/kg×h) or high-dose (HD; 333 μg/kg×h) of Pep19-4LF or saline (vehicle) throughout 4 h after resuscitation. Sham animals were used as control and received saline or high-dose of Pep19-4LF. Data are shown as box and whiskers, showing medians, interquartile range, and full range. The following groups were studied: sham+vehicle (n=11); sham+Pep19-4LF-HD (n=6); HS+vehicle (n=12); HS+Pep19-4LF-LD (n=8); HS+Pep19-4LF-HD (n=8). Statistical analysis was performed using 1-way ANOVA followed by Bonferroni post hoc test. § P<0.05 vs sham+vehicle and *P<0.05 vs HS+vehicle. ANOVA indicated analysis of variance.

FIG. 6: Pep19-2.5 attenuates the organ injury and dysfunction caused by HS. (A) Serum urea, (B) serum creatinine, (C) creatinine clearance (CCr), (D) serum lactate, (E) serum alanine transaminase (ALT), (F) serum aspartate transaminase (AST), (G) serum amylase, and (H) serum lipase of HS or sham-operated rats. All parameters were assessed 4 h subsequent to HS. HS-rats received continuous administration of high-dose (HD; 333 μg/kg×h) of Pep19-2.5 or (HD; 333 μg/kg×h) of Pep19-4LF or saline (vehicle) throughout 4 h after resuscitation. Sham animals were used as control and received saline. Data are shown as box and whiskers, showing medians, interquartile range, and full range. The following groups were studied: sham+vehicle (n=11); HS+vehicle (n=12); HS+Pep19-4LF-HD (n=8); HS+Pep19-2.5-HD (n=8). Statistical analysis was performed using 1-way ANOVA followed by Bonferroni post hoc test. § P<0.05 vs sham+vehicle and *P<0.05 vs HS+vehicle. ANOVA indicated analysis of variance.

FIG. 7: Pep19-4LF attenuates lung inflammation caused by HS. (A) Representative histological images of lung tissue in the studied groups (Sham+vehicle; HS+vehicle; HS+Pep19-4LF). The cells were labelled with a specific marker for macrophages (CD68, brown dots) to demonstrate the infiltration of lung tissue by macrophages, indicating an inflammatory response (200 fold). (B) Quantitative analysis of the number of CD68-positive cells/mm², (C) MPO activity in lungs of HS or sham-operated rats. The scale bar represents 100 μm. All parameters were assessed 4 h subsequent to HS. HS-rats received continuous administration of vehicle (saline) or Pep19-4LF of (333 μg/kg×h) throughout 4 h after resuscitation. Sham animals were used as control and received saline. Data are shown as box and whiskers, showing medians, interquartile range, and full range. The following groups were studied: sham+vehicle (n=4); HS+vehicle (n=6); HS+Pep19-4LF HD (n=6). Statistical analysis was performed using 1-way ANOVA followed by Bonferroni post hoc test. § P<0.05 vs sham+vehicle and *P<0.05 vs HS+vehicle. ANOVA indicated analysis of variance.

FIG. 8: Pep19-4LF attenuates the increase in IL-6 and MCP-1 caused by HS. The serum concentrations of (A) interleukin (IL)-6, (B) monocyte chemotactic protein-1 (MCP-1), (C) IL-10, and (D) C-X-C motif ligand 1 (CXCL1) were determined using a cytometric bead array in sham and HS rats treated with vehicle or Pep19-4LF (333 μg/kg×h) throughout 4 h after resuscitation. All parameters were assessed 4 h subsequent to HS. Data are presented as box and whiskers format, showing medians, interquartile range, and full range. (E) Heat map of measured cytokines (from data in FIG. 8A-D), represented using colour profiles. The following groups were studied: sham+vehicle (n=11); HS+vehicle (n=12), HS+Pep19-4LF-HD (n=8). Statistical analysis was performed using 1-way ANOVA followed by Bonferroni post hoc test. § P<0.05 vs sham+vehicle and *P<0.05 vs HS+vehicle. ANOVA indicated analysis of variance.

FIG. 9: Pep19-4LF attenuates the activation of NF-kB and increases the activation of Akt and eNOS in kidney and liver after HS. The phosphorylation of Ser^{176/180} on IKKα/β (kidney (A), liver (F)), of Ser^{32/36} on IκBα (kidney (B), liver (G), the nuclear translocation of p65 subunit of NF-κB (kidney (C), liver (H)), and Ser⁴⁷³ on Akt (kidney (D), liver (I)), and Ser¹¹⁷⁷ on eNOS (kidney (E), liver (J) of sham and HS rats treated with vehicle or high-dose of Pep19-4LF (333 μg/kg×h) upon resuscitation were determined by Western blotting. Protein expression was measured as relative OD. Data are shown as box and whiskers, showing medians, interquartile range, and full range. The following groups were studied: sham+vehicle (n=4); HS+vehicle (n=4); HS+Pep19-4LF-HD (n=4). Statistical analysis was performed using 1-way ANOVA followed by Bonferroni post hoc test. § P<0.05 vs sham+vehicle and *P<0.05 vs HS+vehicle. ANOVA indicated analysis of variance; OD, optical density.

FIG. 10: Pep19-4LF interacts with heparan sulfate. (A) Inhibitory effect of Pep19-4LF on heparan sulfate-induced TNFα release in human peripheral blood mononuclear cells from healthy donors. Pep19-4LF was added at the indicated weight ratios of the concentrations of heparan sulfate to Pep19-4LF. (B) Red blood cells, obtained from citrated human blood were suspended at a concentration equivalent to 5% of the normal hematocrit. Pep19-4LF and melittin (as control) were added at different concentrations and the supernatants were analyzed for haemoglobin. Results are expressed as the percentage released with respect to sonicated controls (100% release) or controls processed without peptide (0% release). (C) Enthalpy of the Pep19-4LF-heparan sulfate binding. Isothermal calorimetric titration of a 1-4 mM Pep19-4LF solution into a 200 μg/ml heparan sulfate dispersion. The enthalpy changes at each injection were measured and the area under the peak was integrated and plotted against the weight ratio of the concentrations of Pep19-4LF to heparan sulfate. A downward peak corresponds to an exothermic reaction, and an upward peak corresponds to an endothermic reaction.

### BRIEF DESCRIPTION OF THE SEQUENCES

SEQ ID NO: 1 to SEQ ID NO: 3 are the amino acid sequences of three peptides for use in the invention that are 17 amino acids in length.

SEQ ID NO: 4 is the amino acid sequence of a peptides for use in the invention that is 18 amino acids in length.

SEQ ID NOs: 5 to SEQ ID NO: 8 are the amino acid sequences of four peptides for use in the invention that are 19 amino acids in length.

SEQ ID NO: 9 is the amino acid sequence of an exemplary peptide for use in the invention that is 19 amino acids in length. The peptide consisting of this sequence may be referred to herein as Pep19-4LF or Pep4-LF.

SEQ ID NO: 10 is the amino acid sequence of an exemplary peptide for use in the invention that is 20 amino acids in length. The peptide consisting of this sequence may be referred to herein as Pep19-2.5 or Pep2.5.

SEQ ID NO: 11 to SEQ ID NO: 15 are the amino acid sequences of five peptides for use in the invention that are 21 amino acids in length.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting. All publications, patents and patent applications cited herein, whether supra or infra, are hereby incorporated by reference in their entirety.

As used in this specification and the appended claims, the singular forms “a”, “an”, and “the” include plural referents unless the content clearly dictates otherwise. Thus, for example, reference to “a peptide” includes “peptides”, and the like.

### Structural Features of the Peptide

This section sets out the structural features of a peptide for use in the invention.

The primary structure (amino acid sequence) of the peptide is based on the primary structure of an animal LPS-binding protein from Limulus polyphemus, the Limulus anti-LPS factor (LALF). The peptides for use of the invention consist of three regions or domains. The N-terminal is predominantly positively charged, the C-terminal predominantly hydrophobic and the central region is composed of amino acids belonging to different classes.

The peptide is typically at least 17, 18, 19, 20, 21, 22, 23, 24, 25, 26, 27, 28, 29 and up to 30 amino acids in length. It will be appreciated that any of the above listed lower limits may be combined with any of the above listed upper limits to provide a range for the length the peptide. For example, the peptide may be 17 to 26 amino acids in length, 18 to 28 amino acids in length, or 19 to 30 amino acids in length. The peptide is preferably 17 to 21 amino acids in length. The peptide is most preferably 19 or 20 amino acids in length.

The peptide for use in the invention is typically linear and non-cyclic, and may comprise, consist essentially, or consist of 17 to 21 amino acids, wherein the amino acids in positions (1) to (21), counted from the N-terminus, are as follows: (1) G, S or lacking; (2) C or lacking; (3) K or R; (4) K or R; (5) Y or F; (6) K or R; (7) K or R; (8) F, W or L; (9) K, R or lacking; (10) W, L or F; (11) K or R; (12) F, Y or C; (13) K or R; (14) G or Q; (15) K or R; (16) F, L or W; (17) F or W; (18) F, L or W; (19) W or F; (20) C or lacking; (21) G or lacking, wherein when position (9) is lacking, positions (1) and (21) cannot be lacking. The term “lacking” means that no amino acid is present at the specified position. Thus, when positions (1), (2), (20) and (21) are lacking, this means that there is no amino acid present at these positions, therefore the peptide is 17 amino acids in length; when any one of these positions are lacking, the peptide is 18 amino acids; when any two of these positions are lacking, the peptide is 19 amino acids in length; when any three of these positions are lacking, the peptide is 20 amino acids in length; when none of these positions are lacking, the peptide is 21 amino acids in length. When positions (9), (2) and (20) are lacking, the peptide is 18 amino acids in length; when position (9) is lacking and either position (2) or (20) is also lacking, the peptide is 19 amino acids in length; when only position (9) is lacking, the peptide is 20 amino acids in length. The above is applicable throughout the disclosure herein.

The peptide for use in the invention may comprise any positively charged amino acid, preferably arginine (R) and lysine (K), any hydrophobic amino acids, preferably tryptophan (W) and phenylalanine (F) and tyrosine (Y) and, cysteine (C). The number of positively charged amino acids may range from 1 to 5, 3 to 6, 4 to 8 or 5 to 10. Preferably, the number of positively charged amino acids ranges from 7 to 9. The number of hydrophobic amino acids may be 3, 4, 5, 6, 7, 8, 9, 10, 11, 12, 13, 14, or 15. It is preferred that the number of hydrophobic amino acids is 7, 8 or 9. The remaining amino acids can be any amino acid, but preferably the remaining amino acids are polar. The peptide of the invention or a variant thereof may comprise or may have 7 to 9 positively charged amino acids and 7, 8 or 9 hydrophobic amino acids.

The peptide may comprise at least 1, 2, 3, 4, 5 or 6 glycine (G) amino acid residues. Preferably, the peptide comprises 1, 2 or 3 G amino acid residues. The peptide preferably begins and ends with a G amino acid residue. The peptide may comprise none, or at least 1, 2, 3, 4, 5, 6, 7, 8, 9 or 10 lysine (K) or arginine (R) amino acid resides. The peptide may comprise up to 1, up to 2, up to 3, up to 4, up to 5, up to 6, or up to 7 K amino acid residues. The peptide may comprise up to 1, up to 2, up to 3, up to 4, up to 5, up to 6, up to 7, up to 8, or up to 9 R amino acid resides. The peptide preferably comprises 5 K amino acid residues and 3 R amino acid residues. The peptide may comprise at least 1, 2, 3, 4 or 5 phenylalanine (F) amino acid residues. The peptide may comprise up to 1, up to 2, up to 3, up to 4 or up to 5 F amino acid residues. The peptide preferably comprises no more than 4 F amino acid residues. The peptide may comprise none, or at least 1, 2, 3 or 4 leucine (L) amino acid residues. The peptide may comprise up to 1, up to 2 or up to 3 L amino acid residues. The peptide preferably comprises none or 2 L amino acid residues. The peptide may comprise at least 1, 2, 3, 4, or 5 tryptophan (W) amino acid residues. The peptide may comprise up to 1, up to 2 or up to three W amino acid residues. The peptide preferably comprises 1 or 3 W amino acid residues. The peptide may comprise none, or at least 1, 2, 3, 4, or 5 tyrosine (Y) amino acid residues. The peptide may comprise up to 1 or up to 2 Y amino acid residues. The peptide preferably comprises 1 Y amino acid residue. The peptide may comprise at least 1, 2, 3 or 4 cysteine (C) amino acid residues. The peptide may comprise up to 1, up to 2 or up to three C amino acid residues. The peptide preferably comprises 1 C amino residue or does not comprise C amino acid residues. In one embodiment, the peptide may comprise no more than 6 K amino acid residues, no more than four F amino acid residues, and/or no more than three L amino acid residues. In another embodiment, the peptide may comprise no more than 6 K amino acid residues, no more than four F amino acid residues, and/or no more than three W amino acid residues. The peptide of the invention or a variant thereof may comprise or may have at least one G amino acid residue, no more than six K amino acid residues, no more than four F amino acid residues, and/or no more than three L amino acid residues.

For example, the peptide may have any one of the following amino acid sequences:

| | |
|---|---|
| KKFRRLKWKYKGKFWFW; | (SEQ ID NO: 1) |
| KKYRRFRWKFKGKFWFW; | (SEQ ID NO: 2) |
| RRYKKFKWRYRGRFWFW; | (SEQ ID NO: 3) |
| GRRYRRFWKFRGKWFFWG; | (SEQ ID NO: 4) |
| GKKYRRFRWKFKGKWFWFG; | (SEQ ID NO: 5) |
| GRRYRRFRWRFRGRFWFWG; | (SEQ ID NO: 6) |
| GRRYKKFRWKFKGRWFWFG; | (SEQ ID NO: 7) |
| GRRYRRFRWRFRGRFWFWG; | (SEQ ID NO: 8) |
| GKKYRRFRWKFKGKLFLFG; | (SEQ ID NO: 9) |
| GCKKYRRFRWKFKGKFWFWG; | (SEQ ID NO: 10) |
| GCKKFRRFKLKCKQKLWLWCG; | (SEQ ID NO: 11) |
| GCKKFRRLKWKYKGKFWFWCG; | (SEQ ID NO: 12) |
| GCRRYRRFRWKFRGRFWFWCG; | (SEQ ID NO: 13) |
| GCRRYKKFKWRYRGRFWFWCG; | (SEQ ID NO: 14) |
| GCRRFRRFRWRYRGRFWFWCG. | (SEQ ID NO: 15) |

In a preferred embodiment, the peptide for use in the invention may comprise, consist essentially, or consist of 19 amino acids, and wherein the amino acids in positions (1) to (21), counted from the N-terminus, are as follows: (1) G; (2) lacking; (3) K or R; (4) K or R; (5) Y or F; (6) K or R; (7) K or R; (8) F, W or L; (9) K or R; (10) W, L or F; (11) K or R; (12) F, Y or C; (13) K or R; (14) G or Q; (15) K or R; (16) L; (17) F; (18) L; (19) F; (20) lacking; (21) G. An exemplary peptide for use in the invention consists of an amino acid sequence of GKKYRRFRWKFKGKLFLFG (SEQ ID NO: 9). The peptide consisting of this sequence may be referred to herein as Pep19-4LF or Pep4-LF.

In a preferred embodiment, the peptide for use in the invention may comprise, consist essentially, or consist of 20 amino acids, and wherein the amino acids in positions (1) to (21), counted from the N-terminus, are as follows: (1) G; (2) C; (3) K or R; (4) K or R; (5) Y or F; (6) K or R; (7) K or R; (8) F, W or L; (9) K or R; (10) W, L or F; (11) K or R; (12) F, Y or C; (13) K or R; (14) G or Q; (15) K or R; (16) F; (17) W; (18) F; (19) W; (20) lacking; (21) G. An exemplary peptide for use in the invention consists of an amino acid sequence of GCKKYRRFRWKFKGKFWFWG (SEQ ID NO: 10). The peptide consisting of this sequence may be referred to herein as Pep19-2.5 or Pep2.5.

The sequence of a peptide used in the present invention may comprise a variant of the amino acid sequence of SEQ ID NO: 9 or of a peptide comprising the following amino acids at each position counted from the N-terminus, (1) G; (2) lacking; (3) K or R; (4) K or R; (5) Y or F; (6) K or R; (7) K or R; (8) F, W or L; (9) K or R; (10) W, L or F; (11) K or R; (12) F, Y or C; (13) K or R; (14) G or Q; (15) K or R; (16) L; (17) F; (18) L; (19) F; (20) lacking; (21) G, in which modifications, such as amino acid additions, deletions or substitutions are made relative to the sequences specified above.

The sequence of a peptide used in the present invention may comprise a variant of the amino acid sequence of SEQ ID NO: 10 or of a peptide comprising the following amino acids at each position counted from the N-terminus, (1) G; (2) C; (3) K or R; (4) K or R; (5) Y or F; (6) K or R; (7) K or R; (8) F, W or L; (9) K or R; (10) W, L or F; (11) K or R; (12) F, Y or C; (13) K or R; (14) G or Q; (15) K or R; (16) F; (17) W; (18) F; (19) W; (20) lacking; (21) G, in which modifications, such as amino acid additions, deletions or substitutions are made relative to the sequences specified above.

A sequence of a peptide for use in the invention may comprise a variant of the amino acid sequences specified above, in which up to 1, 2 or 3 conservative substitutions are made. Unless otherwise specified, the modifications are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2.

TABLE A1
Chemical properties of amino acids

| | |
|---|---|
| Ala (A) | aliphatic, hydrophobic, neutral |
| Cys (C) | polar, hydrophobic, neutral |
| Asp (D) | polar, hydrophilic, charged (−) |
| Glu (E) | polar, hydrophilic, charged (−) |
| Phe (F) | aromatic, hydrophobic, neutral |
| Gly (G) | aliphatic, neutral |
| His (H) | aromatic, polar, hydrophilic, charged (+) |
| Ile (I) | aliphatic, hydrophobic, neutral |
| Lys (K) | polar, hydrophilic, charged(+) |
| Leu (L) | aliphatic, hydrophobic, neutral |
| Met (M) | hydrophobic, neutral |
| Asn (N) | polar, hydrophilic, neutral |
| Pro (P) | hydrophobic, neutral |
| Gln (Q) | polar, hydrophilic, neutral |
| Arg (R) | polar, hydrophilic, charged (+) |
| Ser (S) | polar, hydrophilic, neutral |
| Thr (T) | polar, hydrophilic, neutral |
| Val (V) | aliphatic, hydrophobic, neutral |
| Trp (W) | aromatic, hydrophobic, neutral |
| Tyr (Y) | aromatic, polar, hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

### General Peptide Features

A “peptide” is used herein in its broadest sense to refer to a compound of two or more subunit amino acids, amino acid analogs, or other peptidomimetics. The term “peptide” thus includes short peptide sequences and also longer polypeptides and proteins. The terms “protein”, “peptide” and “polypeptide” may be used interchangeably. As used herein, the term “amino acid” and “amino acid residue” may be used interchangeably, and refers to either natural and/or unnatural or synthetic amino acids, including both D or L optical isomers, and amino acid analogs and peptidomimetics.

A peptide may be produced by any suitable method, including recombinant or synthetic methods, or semi-synthetic methods for example by combining a recombinant and synthetic production. For example, the peptide may be synthesised directly using standard techniques known in the art, such as Fmoc solid phase chemistry, Boc solid phase chemistry or by solution phase peptide synthesis. A peptide may also be synthesised using in vitro translation of mRNA. Suitable cell-free expression systems include rabbit reticulocyte lysate, wheat germ extract, canine pancreatic microsomal membranes, E. coli S30 extract, and coupled transcription/translation systems such as the TNT-system (Promega). These systems allow the expression of recombinant peptides or proteins upon the addition of cloning vectors, DNA fragments, or RNA sequences containing coding regions and appropriate promoter elements. Alternatively, a peptide may be produced by transforming a cell, typically a bacterial cell, with a nucleic acid molecule or vector which encodes said peptide. A large number of suitable methods exist in the art to produce peptides in appropriate hosts under appropriate culture conditions, such as in a prokaryote, mammalian or insect cell. The produced protein is harvested from the culture medium, lysates of the cultured cells or from isolated (biological) membranes by established techniques. For example, nucleic acid sequences of the peptide can be synthesised by PCR and inserted into an expression vector. Subsequently a suitable host cell may be transfected or transformed with the expression vector. The host cell is then cultured to produce the desired peptide, which is isolated and purified. In an exemplary embodiment, the peptide is synthesised by solid-phase peptide synthesis.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide encodes a peptide for use in the invention and may be provided in isolated or substantially isolated form. By substantially isolated, it is meant that there may be substantial, but not total, isolation of the peptide from any surrounding medium. The polynucleotides may be mixed with carriers or diluents which will not interfere with their intended use and still be regarded as substantially isolated. A nucleic acid sequence which “encodes” a selected peptide is a nucleic acid molecule which is transcribed (in the case of DNA) and translated (in the case of mRNA) into a peptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules may be provided in the form of an expression cassette which includes control sequences operably linked to the inserted sequence, thus allowing for expression of the peptide in vivo. These expression cassettes, in turn, are typically provided within vectors (e.g., plasmids or recombinant viral vectors). Such an expression cassette may be administered directly to a host subject. Alternatively, a vector comprising a polynucleotide may be administered to a host subject. Preferably the polynucleotide is prepared and/or administered using a genetic vector. A suitable vector may be any vector which is capable of carrying a sufficient amount of genetic information, and allowing expression of a peptide for use in the invention.

Expression vectors that comprise such polynucleotide sequences are described. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

Cells that have been modified to express a peptide for use in the invention typically include prokaryotic cells such as bacterial cells, for example E. coli. Such cells may be cultured using routine methods to produce a peptide for use in the invention.

A peptide may be derivatised or modified to assist with their production, isolation or purification. For example, where a peptide is produced by recombinant expression in a bacterial host cell, the sequence of the peptide may include an additional methionine (M) residue at the N terminus to improve expression. As another example, the peptide may be derivatised or modified by addition of a ligand which is capable of binding directly and specifically to a separation means. Alternatively, the peptide may be derivatised or modified by addition of one member of a binding pair and the separation means comprises a reagent that is derivatised or modified by addition of the other member of a binding pair. Any suitable binding pair can be used. For example, where the peptide for use in the invention is derivatised or modified by addition of one member of a binding pair, the peptide may be histidine-tagged or biotin-tagged. Typically the amino acid coding sequence of the histidine or biotin tag is included at the gene level and the peptide is expressed recombinantly in E. coli. The histidine or biotin tag is typically present at either end of the peptide. It may be joined directly to the peptide or joined indirectly by any suitable linker sequence, such as 3, 4 or 5 glycine residues, or a mixture of glycine and serine residues. The histidine tag typically consists of six histidine residues, although it can be longer than this, typically up to 7, 8, 9, 10 or 20 amino acids or shorter, for example 5, 4, 3, 2 or 1 amino acids.

The peptide for use in the invention or a variant thereof may be fused to a further peptide, such as a tag, signal peptide or an antigenic determinant that is known in the art. Such additional sequences may aid with expression and/or purification, increase the solubility of the peptide or be used to target the peptide of interest to an organ or tissue wherein the cells express certain antigens to which the tag bind. For example, the tag may be a histidine tag, human influenza hemagglutinin (HA) tag, FLAG-tag or biotin tag. The tag may be linked to the N or C terminus by a linker. A linker may be used to connect or fuse the peptides. The linker may physically separate the peptides to ensure that neither peptide is limited in their function due to the close vicinity to the other. Depending on what the further peptide is, the linker can be a peptide bond, an amino acid, a peptide of appropriate length, or a different molecule providing the desired features, or any appropriate linker known to the skilled person. For example, peptide linkers can be chosen from the LIP (Loops in Proteins) database (Michalsky et al (2003) Protein Eng Des Sel, (12): 979-985). A linker may be attached to the N- or the C-terminus of the peptide. The linker is preferably located at the N-terminus. In a preferred embodiment, the linker is a lysine, glycine, serine, an ether, ester or a disulphide. Signal peptides are short amino acid sequences capable of directing the peptide or protein to which they are attached to different cellular compartments or to the extracellular space. Antigenic determinants allow for the purification of the fusion peptides via antibody affinity columns.

The N- and C-terminus of the peptide may be derivatized using conventional chemical synthetic methods. The peptides may contain an acyl group, such as an acetyl group. Methods for acylating, and specifically for acetylating the free amino group at the N-terminus are well known in the art. For the C-terminus, the carboxyl group may be modified by esterification with alcohols or amidated to form —CONH₂ or CONHR. Methods of esterification and amidation are well known in the art. In an exemplary embodiment, the peptide is amidated at the C terminus.

A peptide may be provided in a substantially isolated or purified form. That is, isolated from the majority of the other components present in a cellular extract from a cell in which the peptide was expressed. By substantially purified, it will be understood that the peptide is purified to at least 50%, 60%, 70%, 80% or preferably at least 90% homogeneity. Purity level may be assessed by any suitable means, but typically involves SDS-PAGE analysis of a sample, followed by Coomassie Blue detection. A peptide may be mixed with carriers, diluents or preservatives which will not interfere with the intended purpose of the peptide and still be regarded as substantially isolated or purified. Where a peptide is provided in a composition with an additional active component, such as another peptide, each said peptide will individually be purified to a high level of homogeneity prior to mixing in an appropriate ratio for the intended purpose of each. For example, two peptides may be each be purified to at least 90% homogeneity prior to combining in a 1:1 ratio.

A peptide (or mixture thereof) may be provided in lyophilised form, suitable for reconstitution in aqueous solution prior to use. The lyophilised composition has improved stability enabling longer storage of the peptide. A method of preparing a peptide (or mixture thereof) in lyophilised form, comprising freeze-drying said peptide (or mixture) in a suitable buffer, such as Tris-buffered saline (TBS), is provided herein. A peptide is typically substantially purified prior to freeze-drying. The resulting peptide (or mixture) in lyophilised form is also provided. A method of preparing a solution of a peptide (or mixture), comprising providing the peptide (or mixture) in lyophilised form and reconstituting with a suitable carrier or diluent, such as water, is also provided.

### Methods of Treatment/Medical Use

The present invention relates to a method of preventing or treating hemorrhagic shock (HS), or ameliorating symptoms associated with HS in an individual, wherein the method comprises administering to the individual an effective amount of the peptide as described above.

An individual to be treated by the administration of the peptide may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is preferred.

The method of the invention may be for treating HS. In the case of treating, the patient typically has HS, i.e. has been diagnosed as having HS, or is suspected as having the HS, i.e. shows the symptoms of HS. As used herein, the term “treating” includes any of following: the prevention of HS or of one or more symptoms associated with HS; a reduction or prevention of the development or progression of HS or symptoms; and the reduction or elimination of existing HS or symptoms.

The method may be for preventing HS. In this embodiment, the individual may be asymptomatic. As used herein, the term “preventing” includes the prevention of the onset of HS or of one or more symptoms associated with HS.

The method may be for ameliorating the symptoms associated with HS. As used herein, the term “ameliorating” includes the reduction or elimination of existing HS or symptoms.

Therapy and prevention includes, but is not limited to preventing, alleviating, reducing, curing or at least partially arresting symptoms and/or complications resulting from or associated with HS. When provided therapeutically, the therapy is typically provided at or shortly after the onset of a symptom of HS. Such therapeutic administration is typically to prevent or ameliorate the progression of, or a symptom of HS or to reduce the severity of such a symptom or HS. When provided prophylactically, the treatment is typically provided before the onset of a symptom of HS. Such prophylatic administration is typically to prevent the onset of symptoms of HS.

Hemorrhagic shock (HS), also known as hypovolemic shock, is a condition produced by rapid and significant loss of intravascular volume, such as loss of blood, loss of plasma or loss of sodium in the body and consequent intravascular water. The significant loss of intravascular volume may be caused by external or internal bleeding, severe burns, discharge of body fluids through e.g. lesions, diarrhea or vomiting. HS is frequently due to trauma. The loss of intravascular volume may lead to hemodynamic instability, decreases in oxygen delivery, decreased tissue perfusion, cellular hypoxia, organ damage and dysfunction, multiple organ failure (MOF) and death. For instance, HS may cause a decrease in systolic blood pressure, an increased heart rate, a rise in serum lactate and low base deficit, liver injury, renal dysfunction, pancreatic injury and HS-associated inflammation, such as lung inflammation. In trauma-associated HS, the plasma levels of LL-37 in the individual may be elevated.

The parameters indicative of HS can be measured by any suitable methods and assays known in the art. For instance, inflammation can be measured by evaluating the degree of macrophage infiltration (measured as number of CD68-positive cells) and the degree of neutrophil activation (measured as MPO activity) in the lung, and also the consequent release of pro-inflammatory cytokines, such as IL-6 or MCP-1. An increase in the number of macrophages in the lung and in MPO-activity, and an increase in pro-inflammatory cytokines, such as IL-6 or MCP-1, are indications of inflammation. For instance a colorometric assay can be used to determine MPO activity e.g. (react with a solution of o-Dianisidine and H₂O₂ and measuring the rate of change in absorbance spectrophotometrically at 460 nm), or a method described in Pulli B et al (2013) PLOS ONE 8(7): e67976 can be used. The release of cytokines can be measured, e.g. using a cytometric bead array, or a method described in D. Finco et al (2014) Cytokine, 66(2): 143-155.

The measurement of organ injury/dysfunction parameters can be carried out by analysing blood samples. Such parameters may include the levels of serum urea, serum creatinine, serum lactate, creatinine clearance, alanine aminotransferase (ALT), aspartate aminotransferase (AST), amylase and lipase. For instance, significant increases in serum urea and creatinine and a significant decline in creatinine clearance indicate renal dysfunction. Significant increases in alanine aminotransferase (ALT), aspartate aminotransferase (AST), amylase and lipase indicate liver and pancreatic injury. A significant increase in serum lactate indicates global ischemia. Any suitable method in the art can be used to measure these parameters.

Immunoblot analyses can be used to assess nuclear translocation and phosphorylation of factors such as p65, IκBα, IKKα/β, Akt, and eNOS which are part of the Akt/eNOS cell survival pathways and nuclear factor kappa B (NF-κB) pathway. For instance, protein content can be determined on both nuclear and cytosolic extracts using bicinchoninic acid (BCA) protein assay. SDS-PAGE may be used for separation of proteins. Detection of protein may be carried out using any suitable immublotting method known to the skilled person, such as Western blotting. Autoradiography can be used to visualise the bands and densitometric analysis can be used for quantification.

Immunohistochemistry methods are used to fix and stain tissue samples and allow the visualisation of specific cells or factors. The binding of peptide to heparan sulfate can be measured for example using isothermal titration calorimetry.

Exemplary assays for measuring parameters indicative of HS are also described in the Examples.

### Combination Therapies

A peptide may be used in combination with one or more other therapies or agents intended to prevent and/or to treat HS, or to ameliorate the symptoms associated with HS in the same individual. The therapies or agents may be administered simultaneously, in a combined or separate form, to an individual. The therapies or agents may be administered separately or sequentially to an individual as part of the same therapeutic regimen. The other therapy or administration of an agent may be a general therapy aimed at treating or improving the condition of an individual with HS. The other therapy or administration of an agent may be a specific treatment directed at HS or directed at a particular symptom of HS.

For example, treatment may include blood transfusion, such as blood plasma transfusion, platelet transfusion, red blood cell transfusion. Agents may include intravenous crystalloids, colloidal solutions such as albumin and hetastarch, hypertonic saline, dopamine, dobutamine, adrenaline, noradrenaline, artesunate, antibiotics, steroids, prothrombin complex concentrate (factor IX complex) and/or tranexamic acid.

### Effects of Peptide Administration

Administration of the peptide may attenuate the decline in mean arterial pressure during resuscitation after HS. HS-associated organ injury or dysfunction may also be attenuated. The parameters for assessing organ injury/dysfunction include but are not limited to levels of serum urea, serum creatinine, serum lactate, creatinine clearance, alanine aminotransferase (ALT), aspartate aminotransferase (AST), amylase and lipase. Significant increases in serum urea and creatinine and a significant decline in creatinine clearance is indicative of renal dysfunction. Significant increases in alanine aminotransferase (ALT), aspartate aminotransferase (AST), amylase and lipase are indicative of liver and pancreatic injury. A significant increase in serum lactate is indicative of global ischemia. Administration of an effective amount of the peptide may attenuate the HS-associated increases in serum urea and creatinine, ALT, AST, amylase, lipase and serum lactate. The administration of an effective amount of the peptide may also lead to an increase in creatinine clearance.

The administration of the peptide may also have an anti-inflammatory effect and may activate the Akt/eNOS cell survival pathways and may attenuate the activation of the nuclear factor kappa B (NF-κB) pathway. For instance, the administration of the peptide may attenuate lung inflammation.

The peptide may directly interact with/bind to the DAMP heparan sulfate in human mononuclear cells (MNCs). The peptide may also not show haemolytic activity.

At least one of the above effects are observed. Alternatively, all of these effects are observed.

### Administration Routes, Formulations and Dosages

Specific routes, dosages and methods of administration of the peptide for use in the invention may be routinely determined by the medical practitioner. Typically, a therapeutically effective or a prophylactically effective amount of the peptide is administered to the patient. A prophylactically effective amount is an amount which prevents the onset of one or more symptoms of HS. A therapeutically effective amount of the compound is an amount effective to ameliorate one or more symptoms of HS. A therapeutically or prophylactically effective amount of the peptide is administered. The dose may be determined according to various parameters, especially according to the compound used; the age, weight and condition of the patient to be treated; the route of administration; and the required regimen. Again, a physician will be able to determine the required route of administration and dosage for any particular patient.

The peptide can be administered to the patient by any suitable means. The peptide can be administered by enteral or parenteral routes such as via oral, buccal, anal, pulmonary, intravenous, intra-arterial, intramuscular, intraosseous, intraperitoneal, intraarticular, topical or other appropriate administration routes. The peptide is preferably administered intravenously, intramuscularly or intraosseously.

The peptide may be administered in a variety of dosage forms. It may be administered orally (e.g. as tablets, troches, lozenges, aqueous or oily suspensions, dispersible powders or granules), parenterally, subcutaneously, intravenously, intramuscularly, intraosseously, intrasternally, transdermally or by infusion techniques. The peptide may also be administered as a suppository. A physician will be able to determine the required route of administration for each particular patient.

The peptide can be formulated for use with a pharmaceutically acceptable carrier or diluent and this may be carried out using routine methods in the pharmaceutical art. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions. For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Pharmaceutical compositions suitable for delivery by needleless injection, for example, transdermally, may also be used.

A daily dosage for administration to a subject such as a human may range from about 10 μg/kg to about 50 μg/kg, from about 25 μg/kg to about 75 μg/kg, from 50 μg/kg to 100 μg/kg, from about 75 μg/kg to about 150 μg/kg, from about 100 μg/kg to about 250 μg/kg, from about 150 μg/kg to about 500 μg/kg, from about 200 μg/kg to about 750 μg/kg, from about 500 μg/kg to about 1500 μg/kg. Preferably, a typical daily dose is from about 200 μg/kg to about 1400 μg/kg. In one instance, the peptide may be administered continuously for four hours at a typical dose of from about 50 μg/kg per hour to 350 μg/kg per hour.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the peptide in the patient and the duration of treatment desired.

The skilled person and particularly an appropriate physician will be able to identify an appropriate dosage, for instance taking factors such as age, sex, weight, conditions of the patient to be treated, the severity of the disease and the frequency and route of administration and so on into account.

The peptide may be expressed from a polynucleotide. Preferably, such polynucleotides are provided in the form of an expression vector, which may be expressed in the cells of the patient to be treated. The polynucleotides maybe naked nucleotide sequences or be in combination with cationic lipids, polymers or targeting systems. The polynucleotides may be delivered by any available technique. For example, the polynucleotide may be introduced by needle injection, preferably intradermally, subcutaneously or intramuscularly. Alternatively, the polynucleotide may be delivered directly across the skin using a polynucleotide delivery device such as particle-mediated gene delivery. The polynucleotide may be administered topically to the skin, or to mucosal surfaces for example by intranasal, oral, intravaginal or intrarectal administration.

Uptake of polynucleotide constructs may be enhanced by several known transfection techniques, for example those including the use of transfection agents. Examples of these agents includes cationic agents, for example, calcium phosphate and DEAE-Dextran and lipofectants, for example, lipofectam and transfectam.

The invention is illustrated by the following Example:

### Example 1

### Materials and Methods

### Materials

Unless otherwise stated, all compounds were from Sigma-Aldrich Company Ltd (Poole, Dorset, UK). Ringer's Lactate was from Baxter Healthcare Ltd (Deerfield, Ill.). Thiopental sodium was from Archimedes Pharma Limited (Reading, UK).

### Peptide Synthesis

The synthesis and purification of Pep19-4LF and Pep19-2.5 was performed at the Research Center Borstel, Germany as described previously in Tejada G Md et al (2015) Sci Rep, 5:14292. The amino acid sequences of the 19′ mer and 20′ mer are GKKYRRFRWKFKGKLFLFG (Pep19-4LF; SEQ ID NO: 9) and GCKKYRRFRWKFKGKFWFWG (Pep19-2.5; SEQ ID NO: 10). Pep19-4LF was amidated at the C-terminal end and had a purity of >95% as measured by HPLC and MALDI-TOF mass spectrometry (Tejada G Md et al (2015) Sci Rep, 5:14292).

### LL37-ELISA

Plasma-levels of the cathelicidin-derived human AMP LL-37 in patients were quantified using a commercially available ELISA kit (Cambridge Bioscience Ltd, Cambridge, UK) by following the manufacturer/product specific protocol.

### Cytokine Analysis

Concentrations of serum cytokines were determined using a commercially available cytometric bead array (BD Biosciences, San Jose, Calif. or BioLegend, San Diego, Calif.) following the manufacturer/product specific protocol.

### Determination of Myeloperoxidase (MPO) Activity

Lung samples were homogenized in 5 mM phosphate buffer and centrifuged (13,000 g) for 30 minutes at 4° C. Pellets were re-suspended in a 0.5% hexadecyltrimethylammonium bromide 50 mM phosphate buffer solution, sonicated and put in ice for 20 minutes. After 15 minutes of centrifuge (13,000 g at 4° C.), supernatant was allowed to react with a solution of o-Dianisidine (0.167 mg/mL) and H₂O₂ (0.0005%) in 50 mM phosphate buffer. The rate of change in absorbance was measured spectrophotometrically at 460 nm. Myeloperoxidase activity was defined as the quantity of enzyme degrading 1 μmol of peroxide per minute and was expressed in milliunits per gram of wet tissue.

### Immunoblot Analysis

Semi-quantitative immunoblot analyses of nuclear translocation of p65 and the phosphorylation of IκBα, IKKα/β, Akt, and eNOS were carried out in tissue samples as described before in Sordi R et al (2015) Mol Med, 21:563-75. Briefly, kidney and liver samples were homogenized in buffer and centrifuged at 4000 rpm for 5 min at 4° C. To obtain the cytosolic fraction, supernatants were centrifuged at 14000 rpm at 4° C. for 40 min. The pelleted nuclei were re-suspended in extraction buffer and centrifuged at 14,000 rpm for 20 min at 4° C. Protein content was determined on both nuclear and cytosolic extracts using bicinchoninic acid (BCA) protein assay (Thermo Fisher Scientific Inc, Rockford, Ill.). Proteins were separated by 8% sodium dodecyl sulphate polyacrylamide gel electrophoresis (SDS-PAGE) and transferred to a polyvinyldenediflouoride (PVDF) membrane, which was incubated with a primary antibody [rabbit anti-total Akt (1:1000); rabbit anti-Akt pSer⁴⁷³ (1:1000); mouse anti-IκBα pSer^{32/36} (1:1000); mouse anti-total IκBα (1:1000); rabbit anti-IKKα/β pSer^{176/180} (1:1000); rabbit anti-total IKKα/β (1:1000); rabbit anti-NF-κB p65 (1:1000); goat anti-eNOS pSer¹¹⁷⁷ (1:200); rabbit anti-total eNOS (1:200)]. Membranes were incubated with a secondary antibody conjugated with horseradish peroxidase (1:2000) for 30 min at room temperature and developed with ECL detection system. The immunoreactive bands were visualized by autoradiography and the densitometric analysis was performed using Gel Pro Analyzer 4.5, 2000 software (Media Cybernetics, Silver Spring, Md., USA). The membranes were stripped and incubated with β-actin monoclonal antibody (1:5000) and subsequently with an anti-mouse antibody (1:2000) to assess gel-loading homogeneity. Densitometric analysis of the related bands is expressed as relative optical density, and normalized using the related sham-operated band.

### Immunohistochemistry

Lung samples were obtained at the end of the experiment and fixed in formalin for 48 h and immunohistochemistry was performed as described previously in Sordi R et al (2015) Mol Med, 21:563-75. Briefly, lung tissue was embedded in paraffin and processed to obtain 4 μm sections. After deparaffinization, the slides were then incubated with rabbit anti-CD68 antibody ED1 (1:400; catalog no. MCA341R; AbD Serotec) for 1 h at 37° C. and afterwards incubated for 30 min with labelled polymer-HRP antibody. Counterstaining was performed with Harris hematoxylin. Images were acquired using a NanoZoomer Digital Pathology Scanner (Hamamatsu Photonics K.K. Japan) and analyzed using the NDP Viewer software. The numbers of CD68 positive cells were counted in 10 randomly selected fields (200×) in a double-blinded manner by three independent investigators.

### Human Mononuclear Cells Study

Mononuclear cells (MNC) were isolated from heparinized blood samples obtained from healthy donors as described previously in Gutsmann T et al (2010) Antimicrob Agents Chemother, 54(9):3817-24. The cells were re-suspended in medium (RPMI 1640), and their number was equilibrated at 5×10⁶ cells/ml. For stimulation, 200 μl MNC (1×10⁶ cells) was transferred into each well of a 96-well culture plate. Heparan sulfate and the mixtures of heparan sulfate and peptide were pre-incubated for 30 min at 37° C. and added to the cultures at 20 μl per well. The cells were then incubated for 4 h at 37° C. with 5% CO₂. Supernatants were collected after centrifugation of the culture plates for 10 min at 400×g and stored at −20° C. until immunological determination of tumor necrosis factor alpha (TNFα) was carried out with a sandwich enzyme-linked immunosorbent assay (ELISA) using a monoclonal antibody against TNFα (clone 6b; Intex AG, Switzerland) and described previously in detail (Gutsmann T et al (2010) Antimicrob Agents Chemother, 54(9):3817-24).

### Isothermal Titration Calorimetry (ITC)

Microcalorimetric experiments of peptide binding to heparan sulfate were performed on a MSC isothermal titration calorimeter (MicroCal Inc., Northampton, Mass.) at 37° C. as described before in Martin L et al (2015) PLoS One, 10(11):e0143583. Briefly, after thorough degassing of the samples, Pep19-4LF (1 to 4 mM in 20 mM HEPES, pH 7.0) was titrated to a heparan sulfate suspension (200 μg/ml in 20 mM HEPES, pH 7.0). The enthalpy change during each injection was measured by the instrument, and the area underneath each injection peak was integrated (Origin; MicroCal) and plotted against the weight ratio of the concentrations of peptide to heparan sulfate. Titration of the pure peptide into HEPES buffer resulted in a negligible endothermic reaction due to dilution. All experiments were carried out in duplicate.

### Hemolysis Assay

Red blood cells (RBC) were obtained from citrated human blood by centrifugation (1,500×g; 10 min), washed three times with isotonic 20 mM phosphate-NaCl buffer (pH 7.4), and suspended in the same buffer at a concentration equivalent to 5% of the normal hematocrit. Forty-microliter aliquots of this RBC suspension were added to 0.96 ml of Pep19-4LF dilutions prepared in the same isotonic phosphate solution, incubated at 37° C. for 30 min, and centrifuged (1,500×g, 10 min). The supernatants were analyzed spectrophotometrically (with absorbance at 543 nm) for hemoglobin, and results were expressed as the percentage released with respect to sonicated controls (100% release) or controls processed without peptide (0% release).

### Hemorrhagic Shock and Quantification of Organ Injury and Dysfunction

This study was carried out on 46 male Wistar rats (Charles River Ltd, Margate, UK) weighing 230-350 g receiving a standard diet and water ad libitum. Hemorrhagic shock and quantification of organ injury and dysfunction were performed as described previously in Sordi R et al (2017) Ann Surg, 265(2):408-417. Rats were anesthetized by sodium thiopentone (120 mg/kg i.p. for induction, followed by 10 mg/kg i.v. for maintenance). Cannulation of the trachea, femoral artery (for measuring blood pressure), and carotid arteries (for blood withdrawal), jugular vein (for drug administration), and bladder (for collecting urine) were performed. Blood (up to 1 mL/min) was withdrawn via the cannula inserted in the carotid artery in order to achieve a fall in mean arterial pressure (MAP) to 27 to 32 mmHg. Thereafter, MAP was maintained at this level for a period of 90 min either by further withdrawal of blood during the compensation phase or administration shed blood during the decompensation phase. At 90 min after initiation of hemorrhage (or when 25% of the shed blood had to be re-injected to sustain MAP at 27 to 32 mmHg), animals were resuscitated with the remaining shed blood (mixed with 100 IU/mL heparinized saline) (over a period of 5 min) plus a volume of Ringer's lactate identical to the volume of blood spent during decompensation. During the last 3 h of resuscitation, urine was obtained for the estimation of creatinine clearance. Then, blood samples were collected via the carotid artery for measurement of lactate (Accutrend Plus Meter, Roche Diagnostics, West Sussex, UK) and organ injury/dysfunction parameters. Under deep anesthesia, the heart was removed to terminate the experiment. Blood samples were centrifuged to separate serum, which was used for the determination of urea, creatinine, aspartate aminotransferase (AST), alanine aminotransferase (ALT), lipase, amylase and creatine kinase (CK) by an external contract research facility (IDEXX Laboratories Ltd, West Yorkshire, UK) in a blinded fashion. In addition, lung, kidney and liver samples were taken and stored at −80° C. for further analysis. Sham-operated rats were used as control and underwent identical surgical procedures, but without hemorrhage or resuscitation. Blood pressures during the experiment were measured by Powerlab®, and recorded and analyzed by Labchart® (AD instruments Ltd, Dunedin, New Zealand).

### Study Population and Human Outcome Measurements

### Study Setting and Participants

Trauma patients who presented to an urban major trauma centre were recruited to an ongoing, prospective observational cohort study called the ‘Activation of Coagulation and Inflammation in Trauma Study II’ (ACIT-II). This study was originally established in 2008 to investigate the biological mechanisms underlying acute traumatic coagulopathy and the inflammatory response to trauma. Adult trauma patients who require trauma team activation on admission were eligible for inclusion. Exclusion criteria included age under 16 years, transfer from another hospital, arrival-time greater than 120 min from injury, pre-hospital administration of greater than 2000 ml crystalloid, greater than 5% burns, severe liver disease, known bleeding abnormality (including anticoagulant medication), refused consent and vulnerable patients.

### Patient Selection

Trauma patients for this study (n=47) were identified from the available ACIT-II cohort based on their Injury Severity Score (ISS) and blood product requirements during resuscitation. Patients were included if they had an ISS score greater than or equal to 16. Trauma Haemorrhage patients were defined as patients who received greater than or equal to two units of packed red blood cells (PRBC) on admission. Control patients (No Haemorrhage) received no PRBCs during the first 24 hours of their admission. We subsequently excluded patients with a head Abbreviated Injury Severity (AIS) score greater than three in order to ensure the groups were not skewed on the basis of severe traumatic brain injury. Furthermore, we included healthy volunteers (n=10) as a control group.

### Sample and Data Collection

Admission data were collected on patient demographics, mechanism of injury, blood product use and baseline physiology. Arterial blood gas analysis for base deficit (BD) and lactate was performed during the trauma team resuscitation as part of normal care processes. Admission bloods were drawn within 2 hours of injury. Whole blood was collected in 4.5 ml citrated vacutainer tubes and centrifuged at 3400 rpm for 10 minutes. The plasma supernatant was centrifuged again at the same settings, and the double-spun plasma was subsequently stored in aliquots at −80° C. Patient outcomes including 28-day mortality, ventilator days, critical care and hospital length of stay were recorded.

### Experimental Design

The following groups were studied: Sham+vehicle (n=11), sham+Pep19-4LF (n=6), HS+vehicle (n=12), HS+Pep19-4LF-LD (n=8), HS+Pep19-4LF-HD (n=8), HS+Pep19-2.5-HD (n=8). Rats were administered vehicle (saline 1.5 ml/kg/h) or Pep19-4LF (low dose (LD)=66 μg/kg×h; high dose (HD)=333 μg/kg×h) or Pep19-2.5 (high dose (HD)=333 μg/kg×h) continuously for 4 h after resuscitation using infusion pump for rodents (PHD2000, 70-2000; Harvard apparatus Massachusetts, U.S). 11 rats were excluded from data analysis due to surgical/technical issues (n=5) prior to onset of HS and death during resuscitation (n=6). The doses of Pep19-4LF and Pep19-2.5 used in this study were based on efficacy seen in previous in vitro and in vivo studies (Schuerholz T et al (2013) Crit Care, 17(1):R3; Tejada G Md et al (2015) Sci Rep, 5:14292; Martin L et al (2015) PLoS One, 10(5):e0127584; Martin L et al (2015) PLoS One, 10(11):e0143583).

### Statistics

Unless otherwise stated, the data is expressed as median and standard error or described in box and whisker format showing medians, interquartile ranges and full ranges of n observations, where n represents the number of animals/experiments studied. Statistical analysis was carried out using Prism 6 for Mac OS X (GrapPad, San Diego, Calif., USA). The distribution of the data was assessed using D'Agostino's K-squared test or Kolmogorov-Smirnov test. Unless otherwise stated, normal distributed data were assessed by 1 or 2-way analysis of variance followed by Bonferroni post hoc test. Unless otherwise stated, not normally distributed data were analyzed with a non-parametric test (Kruskal-Wallis followed by Dunn test). A p<0.05 was considered to be significant.

### Results

### Plasma Concentrations of the Host Defence AMP Cathelicidin LL-37

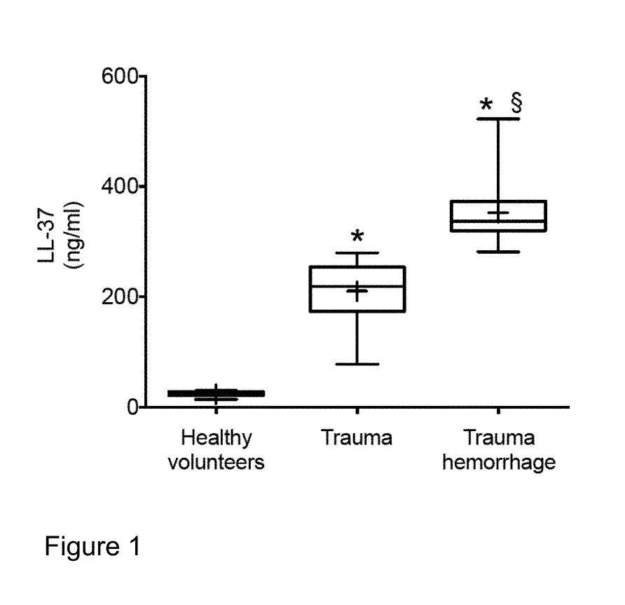

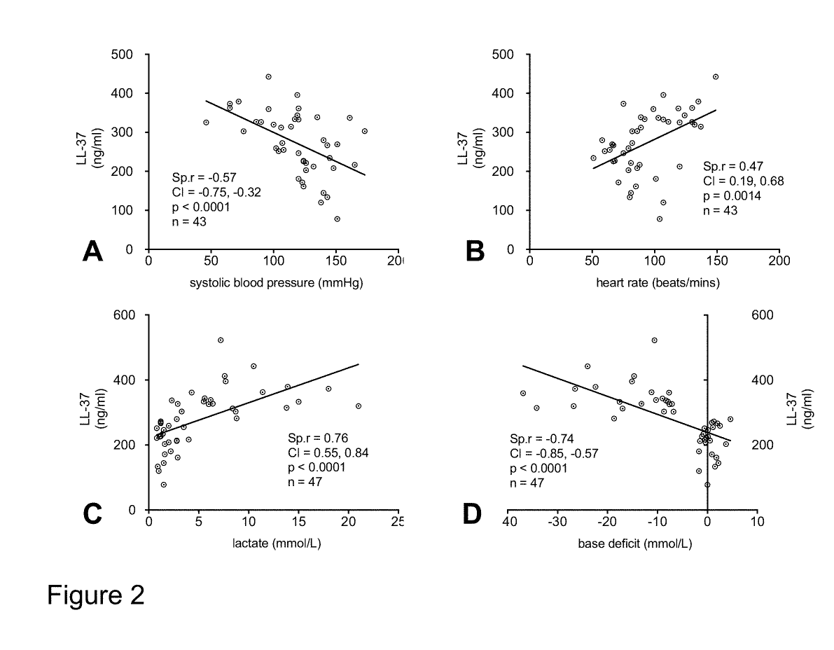

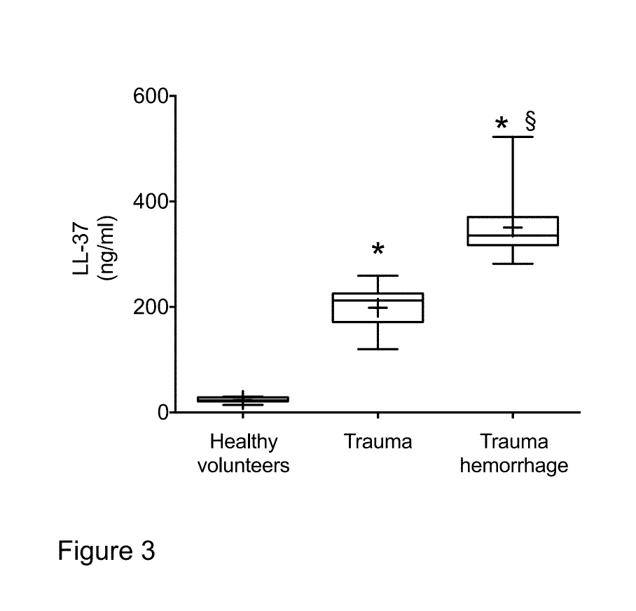

FIG. 1 shows plasma concentrations of LL-37 in healthy volunteers and trauma patients recruited from an urban major trauma center. The median age of the healthy volunteers were 47 (32-53) years with 80% male. Further demographic and clinical parameters of trauma patients are described in Table 1. Admission blood samples of trauma patients were obtained within 2 h of injury (Table 1). When compared to healthy volunteers, trauma patients (n=24) and trauma hemorrhage patients (n=23) (defined as patients who received greater than or equal to two units of packed red blood cells on admission) showed significantly higher plasma levels of LL-37. When compared to trauma patients, the plasma levels of LL-37 were significantly higher in trauma hemorrhage patients (FIG. 1). Increased levels were associated (at time of admission) with (a) low systolic blood pressure, (b) high heart rate, (c) high lactate, and (d) low base deficit (FIG. 2). Moreover, a subgroup analysis with focus on patients with an Injury Severity Score (ISS) of >24 was performed (FIG. 3). Applying this threshold to the cohort resulted in an equivalent median ISS between the groups (Table 2). Notably, this subgroup analysis shows different LL-37 serum concentrations between trauma patients and trauma hemorrhage patients (FIG. 3).

TABLE 1
Admission Characteristics & Outcomes

| | | |
|---|---|---|
| Trauma | Trauma | |
| No Hemorrhage | Hemorrhage | P-value* |

Admission Characteristics

| | | | |
|---|---|---|---|
| N | 24 | 23 | — |
| Age (years) | 43 (35-49) | 32 (24-51) | 0.190 |
| Male (%) | 24 (100) | 16 (69.6) | 0.003^{‡} |
| Blunt Mechanism (%) | 23 (95.8) | 15 (65.2) | 0.008^{‡} |
| Time Injury-Sample | 94 (76-108) | 100 (59-116) | 0.686 |
| (min) | | | |
| Admission HR | 81 (70-88) | 130 (112-141) | <0.001 |
| (beats/min) | | | |
| Admission SBP | 133 (122-145) | 85 (50-113) | <0.001 |
| (mmHg) | | | |
| Admission Base | 0 (−2-1) | 11 (8-22) | <0.001 |
| Deficit (mmol/L | | | |
| Admission Lactate | 2 (1-3) | 8 (6-11) | <0.001 |
| (mmol/L) | | | |
| Pre-baseline PRBC | 0 (0-0) | 1 (0-3) | <0.001 |
| (units) | | | |
| 24 hr PRBC (units) | 0 (0-0) | 10 (5-12) | <0.001 |
| ISS (points) | 23 (20-29) | 29 (25-41) | 0.004 |
| AIS Head (points) | 0 (0-0) | 0 (0-2) | 0.371 |
| AIS Chest (points) | 4 (3-5) | 3 (2-5) | 0.904 |
| AIS Abdomen (points) | 2 (0-3) | 2 (0-3) | 0.444 |
| AIS Extremity (points) | 2 (0-4) | 3 (0-5) | 0.097 |

Outcomes

| | | | |
|---|---|---|---|
| 28-day mortality (%) | 0 (0) | 6 (26.1) | 0.007^{‡} |
| Ventilator Days (days) | 0 (0-2) | 3 (3-7) | <0.001 |
| Critical Care LOS | 0 (0-6) | 9 (4-20) | <0.001 |
| (days) | | | |
| Hospital LOS (days) | 15 (8-38) | 18 (4-51) | <0.773 |

HR, Heart Rate;
SBP, Systolic Blood Pressure;
PRBC, Packed Red Blood Cells;
ISS, Injury Severity Score;
AIS, Abbreviated Injury Severity Score;
LOS, Length of Stay.
Median (interquartile range) reported unless specified.
*Trauma Hemorrhage vs. No Hemorrhage;
Mann-Whitney U-test unless ^{‡}Chi-Squared test.

TABLE 2
Subgroup analysis (patients ISS >24)

| | | |
|---|---|---|
| Trauma | Trauma | P- |
| No Hemorrhage | Hemorrhage | value* |

Admission Characteristics

| | | | |
|---|---|---|---|
| N | 11 | 21 | |
| Age (years) | 46 (37-52) | 32 (24-51) | 0.254 |
| Male (%) | 11 (100) | 14 (66.7) | 0.030^{‡} |
| Blunt Mechanism (%) | 11 (100) | 14 (66.7) | 0.030^{‡} |
| Time Injury-Sample | 100 (90-110) | 102 (76-116) | 0.845 |
| (min) | | | |
| Admission HR | 85 (76-101) | 126 (112-135) | 0.002 |
| (beats/min) | | | |
| Admission SBP (mmHg) | 138 (126-147) | 84 (50-113) | <0.001 |
| Admission Base Deficit | 0 (−1-2) | 11 (9-19) | <0.001 |
| (mmol/L) | | | |
| Admission Lactate | 2 (1-2) | 8 (6-11) | <0.001 |
| (mmol/L) | | | |
| Pre-baseline PRBC | 0 (0-0) | 2 (0-3) | 0.001 |
| (units) | | | |
| 24 hr PRBC (units) | 0 (0-0) | 10 (7-12) | <0.001 |
| ISS (point) | 29 (29-34) | 29 (25-41) | 0.775 |
| AIS Head (point) | 0 (0-0) | 0 (0-0) | 0.457 |
| AIS Chest (point) | 5 (4-5) | 4 (2-5) | 0.144 |
| AIS Abdomen (point) | 2 (0-3) | 2 (0-3) | 0.696 |
| AIS Extremity (point) | 2 (0-4) | 3 (0-5) | 0.208 |

Outcomes

| | | | |
|---|---|---|---|
| 28-day mortality (%) | 0 | 6 (28.6) | 0.049^{‡} |
| Ventilator Days (days) | 2 (0-6) | 3 (3-7) | 0.070 |
| Critical Care LOS (days) | 2 (0-6) | 9 (4-13) | 0.011 |
| Hospital LOS (days) | 21 (10-35) | 18 (4-49) | 0.938 |

HR, Heart Rate;
SBP, Systolic Blood Pressure;
PRBC, Packed Red Blood Cells;
ISS, Injury Severity Score;
AIS, Abbreviated Injury Severity Score;
LOS, Length of Stay.
Median (interquartile range) reported unless specified.
*Trauma Hemorrhage vs. No Hemorrhage;
Mann-Whitney U-test unless ^{‡}Chi-Squared test.

Pep19-4LF and Pep19-2.5 Attenuate the Decline in Blood Pressure During Resuscitation after HS

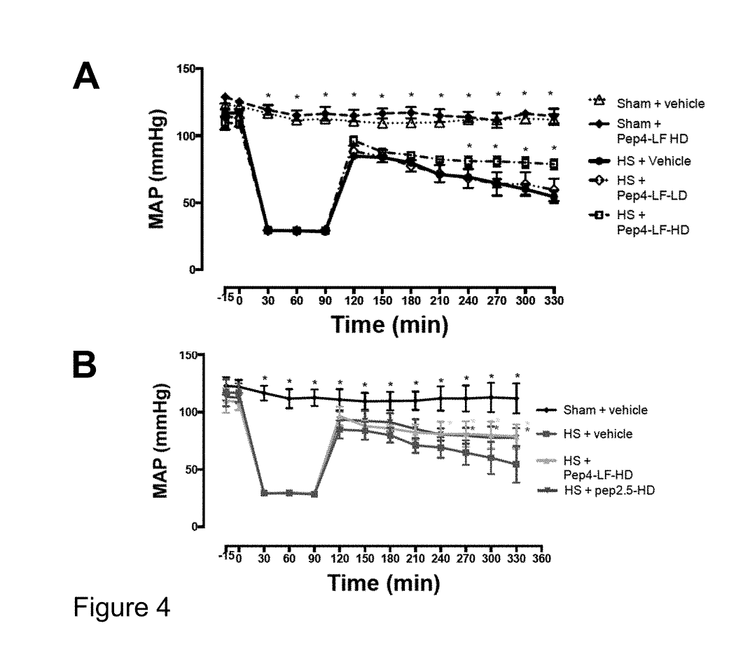

When compared to sham-operated rats, HS-rats treated with vehicle showed a significant decline in mean arterial pressure (MAP) after resuscitation (FIG. 4). Intravenous administration of high-dose Pep19-4LF or Pep19-2.5 significantly attenuated the decline in MAP observed during the resuscitation period in HS-rats, while the low dose of Pep19-4LF had no effect. In contrast, the high dose of Pep19-4LF had no effect of MAP in sham-operated rats (FIG. 4).

### Pep19-4LF and Pep2.5 Attenuate the Organ Injury and Dysfunction Caused by HS

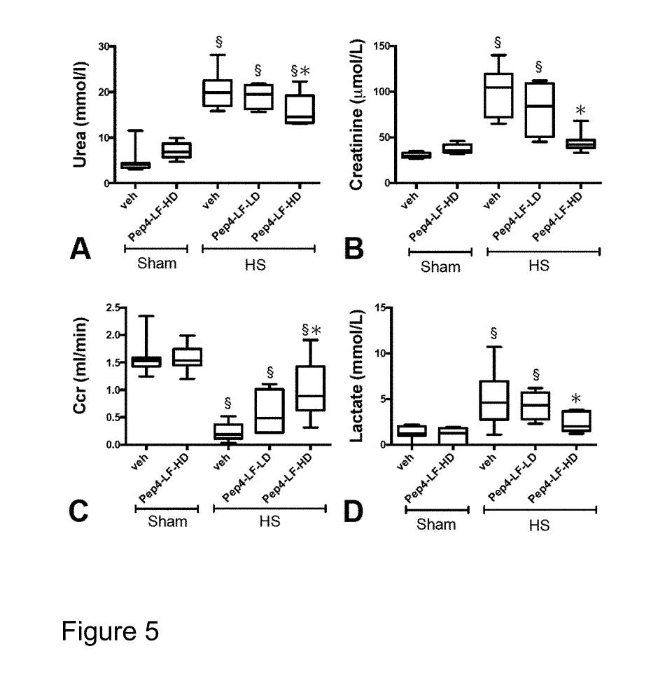

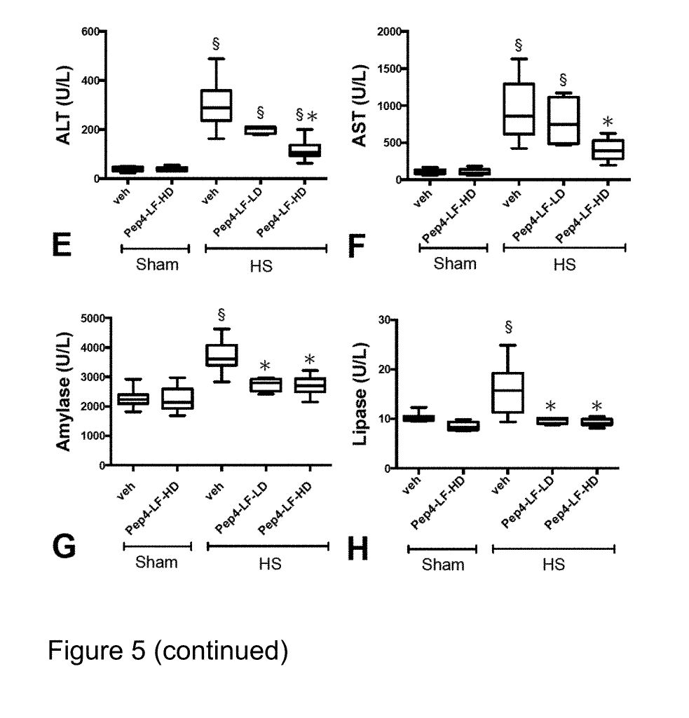

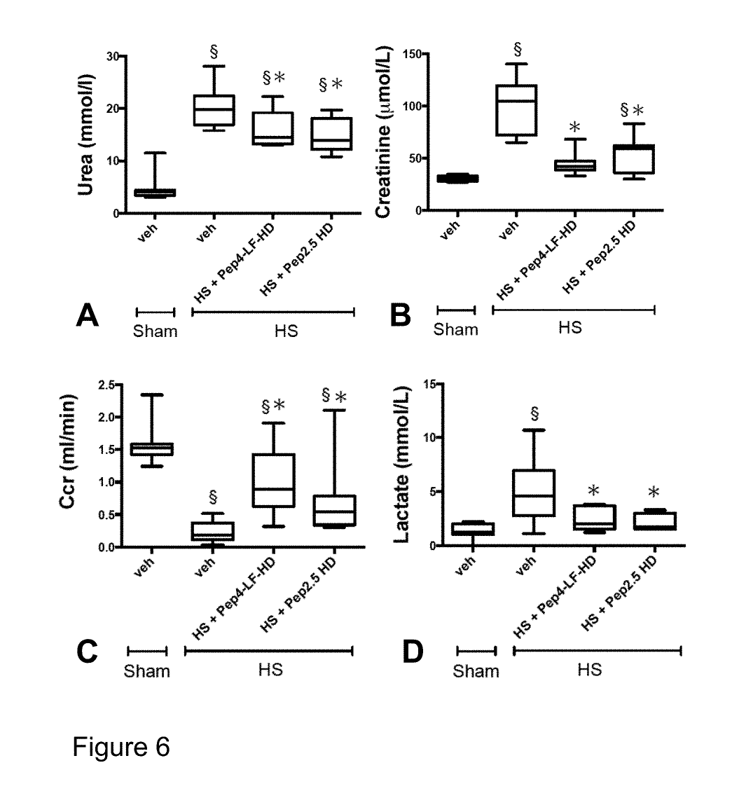

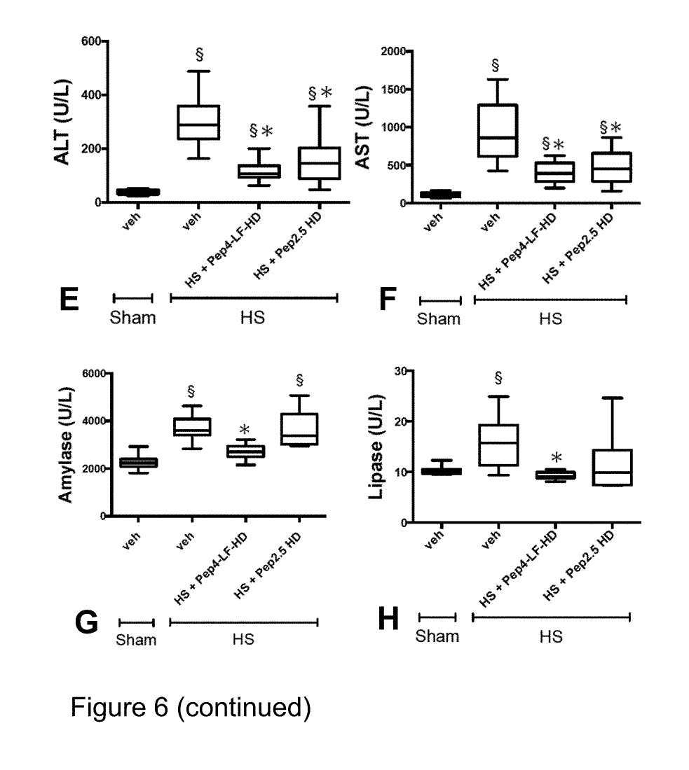

When compared to sham-operated rats, rats subjected to HS treated with vehicle exhibited a renal dysfunction, as indicated by significant increases in serum urea (FIG. 5A) and creatinine (FIG. 5B), and a significant decline in creatinine clearance (FIG. 5C). HS-rats also exhibited significant increases in alanine aminotransferase (ALT), aspartate aminotransferase (AST), amylase and lipase, indicating the development of liver and pancreatic injury, respectively (FIGS. 5E-G). In addition, HS-rats exhibited a significant increase in serum lactate, indicating global ischemia (FIG. 5D). When compared to vehicle-treated rats, intravenous administration of high-dose Pep19-4LF or Pep19-.2.5 significantly attenuated the organ injury and dysfunction as well as the rise in lactate caused by HS (FIGS. 5 & 6). Treatment with low dose Pep19-4LF attenuated the increases in serum creatinine, ALT, amylase, and lipase, but did not attenuate the increases in urea, creatinine clearance, lactate or AST caused by HS (FIG. 5).

### Pep19-4LF Attenuates Lung Inflammation Caused by HS

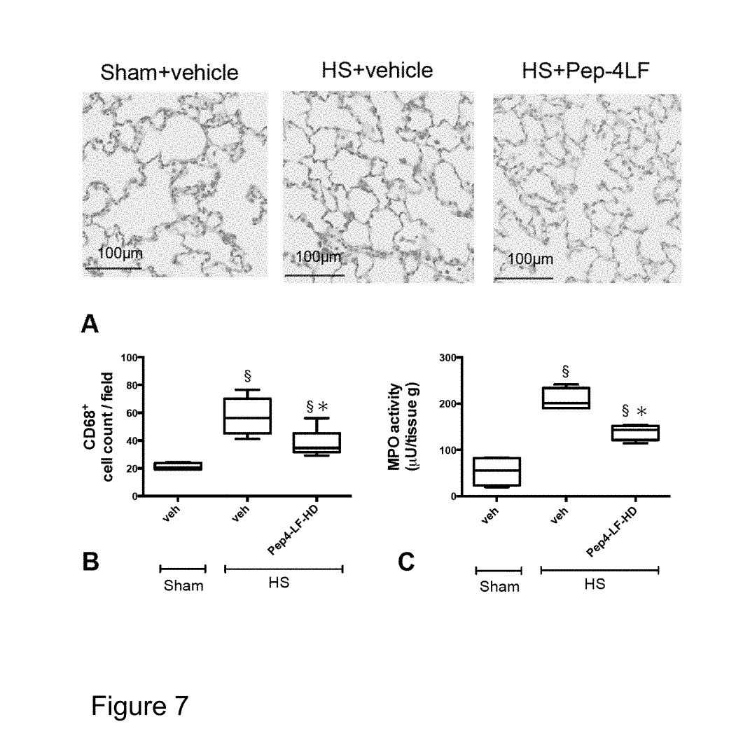

The effects of Pep19-4LF on lung inflammation measured as recruitment of macrophages (CD68-positive cells) and neutrophil activation (MPO activity) into the lung was investigated. When compared to sham-operated rats, we found a significant increase in CD68-positive cells and MPO-activity in lungs of HS-rats treated with vehicle (FIG. 7). Treatment of HS-rats with Pep19-4LF significantly attenuated the recruitment of macrophages and neutrophil activation caused by HS (FIG. 7). FIG. 7 shows representative histological images of lung tissue in the studied groups (Sham+vehicle; HS+vehicle; HS+Pep19-4LF). The cells were labelled with a specific marker for macrophages (CD68, brown dots) to demonstrate the infiltration of lung tissue by macrophages, indicating an inflammatory response.

### Pep19-4LF Attenuates the Increase in Interleukin (IL)-6 and Monocyte Chemotactic Protein-1 (MCP-1) Caused by HS

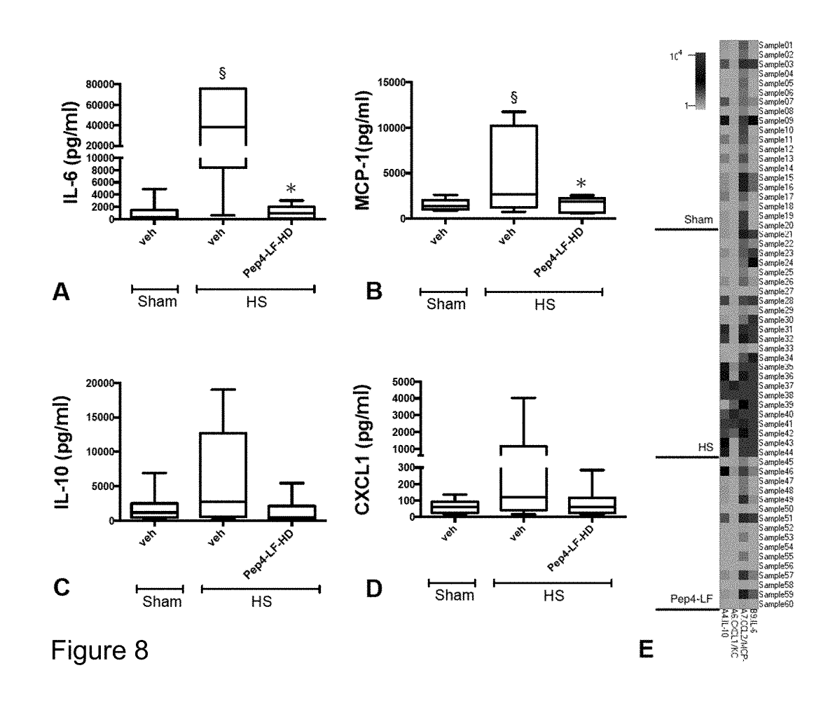

The effects of Pep19-4LF on the formation of pro- and anti-inflammatory cytokines caused by HS was investigated. When compared to sham-operated rats, HS-rats treated with vehicle developed a significant increase in serum IL-6 and MCP-1 (FIGS. 8A & B). In HS-rats, we also observed increases in serum IL-10 and C-X-C motif ligand 1 (CXCL1), but these effects were not significant (FIGS. 8C & D). Treatment of HS-rats with Pep19-4LF abolished the increases in IL-6, MCP-1, IL-10, and CXCL1 caused by HS (FIG. 8).

### Pep19-4LF Attenuates the Activation of NF-κB (Liver and Kidney) Caused by HS

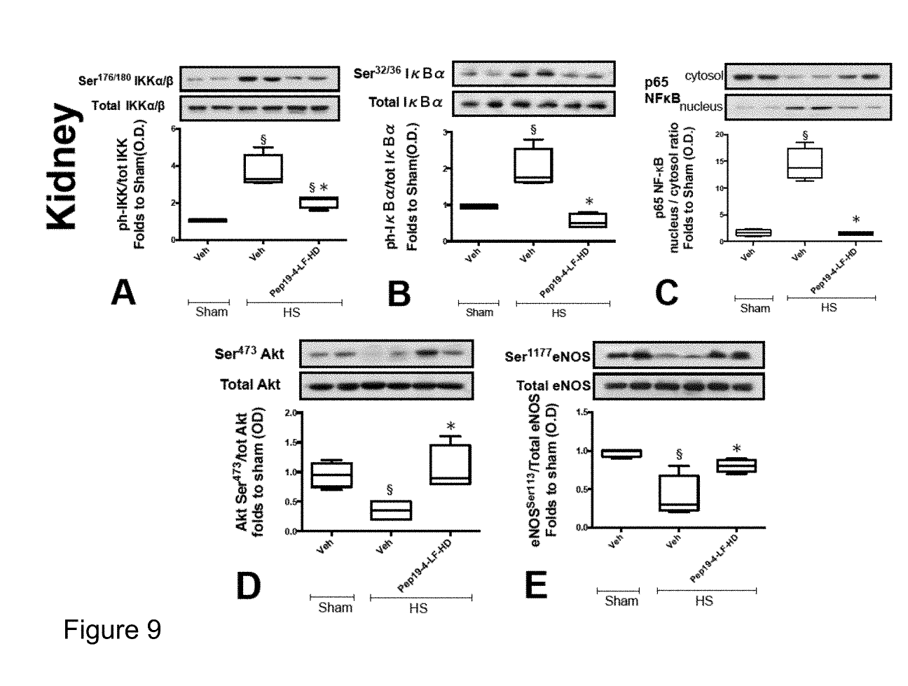

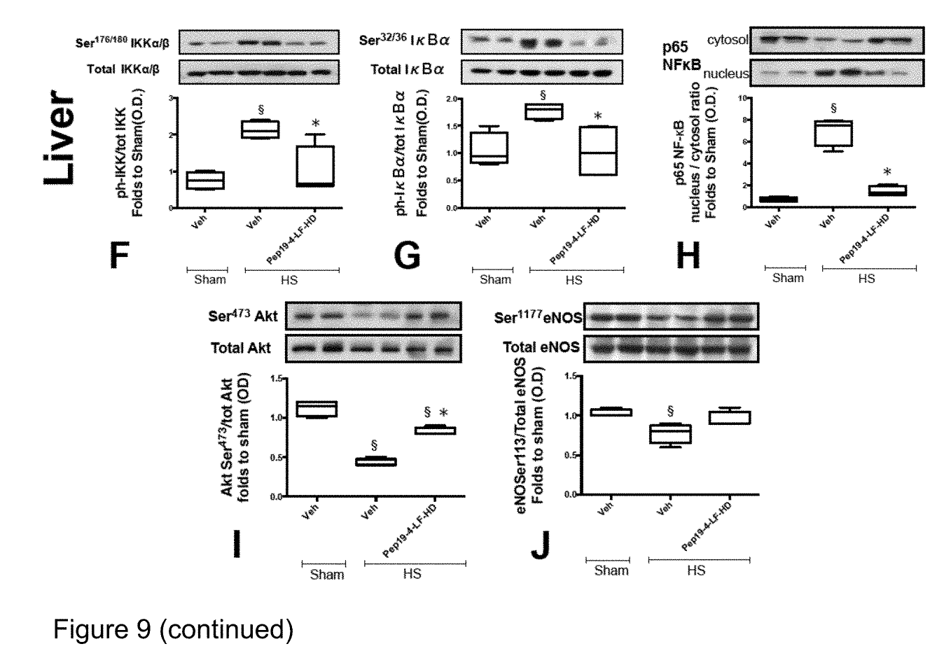

Having shown that Pep19-4LF significantly attenuates kidney dysfunction and liver injury caused by HS, the potential mechanism(s) underlying the observed beneficial effects of high dose of Pep19-4LF was explored. When compared to sham-operated rats, HS-rats treated with vehicle exhibited a significant increase in phosphorylation of IκB kinase a and β (IKKα/β), which is essential for IκB phosphorylation (FIGS. 9A & 8F). Following HS-induced phosphorylation of IκB (FIGS. 9B & 8G), the p65 subunit of NF-κB is freed to translocate to the nucleus, as shown in FIGS. 9C and 9H. The intravenous administration of high-dose Pep19-4LF attenuated the phosphorylation of IKKα/β on Ser^{176/180}, and of IκBα on Ser^{32/36} and, accordingly, the nuclear translocation of the NF-κB subunit p65 in both liver and kidney (FIGS. 9A, 9B, 9C, 9F, 9G & 9H).

Pep19-4LF Increases Activation of Akt and eNOS in Kidney and Liver after HS

As activation of the Akt-survival pathway is known to reduce HS-induced organ dysfunction (Sordi R et al (2017) Ann Surg 265(2):408-417; Sordi R et al (2015) Mol Med, 21:563-75), it was investigated whether the high dose of Pep19-4LF activates Akt in kidney and liver of HS-rats. When compared to sham-operated rats, HS-rats treated with vehicle showed a significant reduction in the phosphorylation of Akt on Ser⁴⁷³ in both kidney and liver (FIGS. 9D & 9I). This effect was associated with a reduction in the Akt-mediated eNOS phosphorylation at Ser¹¹⁷⁷. In contrast, treatment of HS-rats with Pep19-4LF attenuated the decline in Akt phosphorylation on Ser⁴⁷³ and of eNOS phosphorylation on Ser¹¹⁷⁷ in kidney and liver, when compared to HS rats treated with vehicle (FIGS. 9E & 9J).

### Pep19-4LF Inhibits the Heparan Sulfate-Induced TNFα Secretion in Human Peripheral Blood Mononuclear Cells

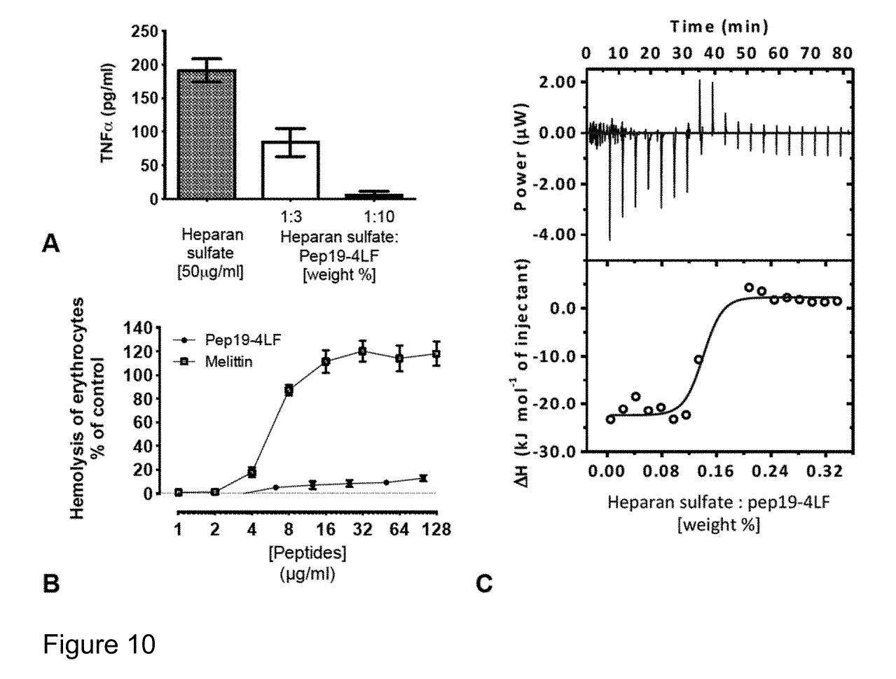

As discussed above, traumatic injury and trauma-associated HS result in the release of a variety of endogenous TLR ligands, including heparan sulfate (Rahbar E et al (2015) Journal of Translational Medicine, 13:117). It is observed here that heparan sulfate stimulates the release of TNFα from human MNCs, and that this effect is reduced/abolished in a concentration-dependent manner by Pep19-4LF (FIG. 10A).

### Pep19-4LF Exhibits Binding to Heparan Sulfate

To gain a better understanding of the mechanism(s) by which Pep19-4LF reduces the formation of TNFα in human MNC challenged with heparan sulfate, the potential binding of Pep19-4LF to heparan sulfate by isothermal titration calorimetry was investigated. There was an exothermic reaction between the two reactants, running into a saturation of binding at higher mass ratios (FIG. 10C). The rather steep slope of the sigmoidal curve indicates distinct binding of the reaction partners and may explain that the binding epitopes of heparan sulfate to receptors (i.e. TLR4) are hidden by the peptide.

Pep19-4LF does not Show Hemolytic Activity

Finally, possible cytotoxic effects of Pep19-4LF were studied in the hemolysis assay with RBC as sensitive target cells for cytotoxicity. Pep19-4LF caused no considerable degree of hemolysis in concentrations of up to 100 μg/ml (FIG. 10B).

### DISCUSSION

We report here for the first time that trauma leads (within 2 h) to a significant increase in the plasma levels of the host-defence/antimicrobial peptide LL-37. Most notably, the highest levels of LL-37 were found in patients with trauma complicated by severe hemorrhage (FIG. 1) and were associated with low systolic blood pressure, high heart rate, high lactate and low base deficit.

Using a reverse translational approach, we investigated whether pharmacological intervention with synthetic host-defence/antimicrobial peptides attenuates the MOF associated with HS in rats. As the therapeutic use of LL37 in man is limited by its systemic toxicity in therapeutic doses (Hancock R E W et al (2006) Nat Biotechnol, 24(12):1551-7; Zhang L & Falla T (2006) Expert Opin Pharmacother, 7(6):653-663), we synthesized Pep19-4LF, which does not cause any significant adverse effects (hemolysis) in the doses used (FIG. 10B). The administration of both Pep19-4LF and Pep19-2.5 significantly attenuated the fall in blood pressure (FIG. 4) as well as the rise in serum lactate caused by HS (FIGS. 5D & 6D). Thus, Pep19-4LF and Pep19-2.5 reduce the delayed vascular decompensation and organ/tissue ischemia probably due to increased microvascular perfusion secondary to increased perfusion pressure. Moreover, Pep19-4LF and Pep19-2.5 significantly attenuated the liver injury, renal dysfunction and pancreatic injury caused by HS (FIGS. 5 & 6). As neutrophils and macrophages play an important role in HS-associated lung inflammation, we evaluated the degree of macrophage infiltration (measured as number of CD68-positive cells) and the degree of neutrophil activation (measured as MPO-activity) in the lung. HS resulted in a significant increase in the number of macrophages in the lung as well as a significant increase in MPO-activity, both of which was attenuated by the treatment of HS-rats with Pep19-4LF (FIG. 7). Neutrophils and macrophages release (via degranulation) pro-inflammatory cytokines, such as IL-6 or MCP-1 which importantly contribute to acute lung injury/inflammation (Dewar D et al (2009) Injury, 40:912-918). The pro-inflammatory cytokines IL-6 and MCP-1 are important mediators of alterations associated with organ dysfunction and even lethality following HS and resuscitation (Jarrer D et al (1999) Int. J. Mol. Med, 4:575-583; Sordi R et al (2015) PLoS One, 10(4):e0122096; Bahrami S et al (1997) Am. J. Physio, 272:H2119; Zhang Y et al (2014) Shock, 42(3):218-27). For instance, a monoclonal antibody against IL-6 reduces the organ dysfunction and inflammation caused by HS. Indeed, we report here that Pep19-4LF also attenuates the rise in serum IL-6 and MCP-1 caused by HS in the rat (FIG. 8).

There is good evidence that PAMPs and DAMPs released during trauma-HS interact with Toll-like receptors (i.e. TLR2,4) resulting in activation of NF-κB (Tang D et al (2012) Immunol Rev, 249(1):158; Pradeu T & Cooper E (2012) Front Immunol, 3:287; Martin L et al (2016) Biomed Res Int, 2016:3758278). Indeed, we observed a significant increase in (a) the degree of phosphorylation of IKKα/β on Ser^{176/180} and (b) of IκBα on Ser^{32/36}, thus resulting in (c) increased nuclear translocation of NF-κB subunit p65 (FIG. 9) in liver/kidneys of rats with HS. This activation of NF-κB in key target organs was attenuated in HS-rats treated with Pep19-4LF during resuscitation. IκBα masks the nuclear localization signals of NF-κB proteins and sequesters NF-κB as an inactive complex in the cytoplasm, thereby inhibiting NF-κB (Jacobs M D & Harrison S C (2008) Cell, 95:749-758; Senftleben U & Karin M (2002) Crit Care Med, 30(Suppl1):S18-S26). Signal-induced proteolytic degradation of IκBα, which has been phosphorylated by icB kinases (IKKα/β) liberates NF-κB to translocate to the nucleus. Subsequently, NF-κB activates the transcription of a number of genes involved in producing pro-inflammatory cytokines and chemokines known to result in the transcription of a multitude of pro-inflammatory cytokines, chemokines and proteins that are widely implicated in the pathophysiology of MOF (Sordi R et al (2015) Mol Med, 21:563-75; Sordi R et al (2017) Ann Surg, 265(2):408-417). Thus, the organ protective effects of Pep19-4LF in HS are associated with a significant reduction in the activation of the NF-κB pathway, which in turn accounts for the reduced formation of IL-6 and MCP-1.

We also investigated the effects of HS with or without Pep19-4LF on the degree of activation of the Akt-survival pathway (FIG. 9). When compared to sham rats, HS rats treated with vehicle showed a significantly decreased phosphorylation of Akt on Ser⁴⁷³ (indicating reduction in activity of this kinase) in kidney and liver, which makes these organs less resistant to organ injury. In contrast, Pep19-4LF attenuated the decline in Akt-activation caused by HS (FIGS. 9 D & I). Akt is a member of the phosphoinositide-3-kinase (PI3K) signal transduction enzyme family. When activated (phosphorylated on Ser⁴⁷³) by its upstream regulator PI3K, Akt controls inflammatory response, chemotaxis and apoptosis (Cantley (2002) Science, 296:1655-1657). Most notably, activation of the Akt-survival pathway reduces organ injury in many conditions associated with ischemia/inflammation including ventilation-induced lung injury, sepsis-induced organ dysfunction, myocardial infarction, and HS-induced organ dysfunction (Shu Y et al (2014) J Trauma Acute Care Surg, 76:1417-1424; Khan A I et al (2013) Dis Model Mech, 6(4): 1021-30; Coldewey S M et al (2013) Dis Model Mech, 6(4):1031-42; Cai Z & Semenza G L (2004) Circulation, 109(17):2050-3; Sordi R et al (2017) Ann Surg 265(2):408-417). Moreover, activation of Akt results in phosphorylation and activation of eNOS at Ser¹¹⁷⁷ that enhances the formation of small amounts of NO, which is pivotal for the preservation of microvascular perfusion and, hence, reducing organ injury (Cabrales P et al (2009) Resuscitation, 80(6):707-12; Khan A I et al (2013) Dis Model Mech, 6(4): 1021-30; Nandra K K et al (2013) Dis Model Mech, 6(3):701-9). We report here that the degree of eNOS phosphorylation on Ser¹¹⁷⁷ is significantly higher in HS-rats treated with Pep19-4LF indicating activation of eNOS and enhanced formation of NO in the microcirculation at least of kidney and liver. We propose that the enhanced formation of NO by eNOS in HS-rats treated with Pep19-4LF contributes to improved microcirculatory perfusion resulting in better tissue oxygenation and lower lactate levels (FIG. 5D). Thus, the organ protective effects of Pep19-4LF in HS are associated with a significant activation of the Akt/eNOS survival pathway.

Traumatic injury and trauma-associated HS result in the release of a variety of endogenous TLR ligands, including heparan sulfates (Tang D et al (2012) Immunol Rev, 249(1): 158; Pradeu T & Cooper E (2012) Front Immunol; 3:287; Martin L et al (2016) Biomed Res Int, 2016:3758278; Horst K et al (2016) Eur J Trauma Emerg Surg, 42:67-75). Moreover, the degradation of the glycocalyx (and subsequent liberation of heparan sulfates) induces remote organ injury after trauma/hemorrhagic shock (Wu H et al (2010) J Am Soc Nephrol, 21(11):1878-90; Sodhi C P et al (2015) J Immunol, 194(10):4931-9; Torres Filho I P et al. (2016) Am J Physiol Heart Cire Physiol, 310(11):H1468-1478; Levy R M et al (2007) Am J Physiol Regul Integr Comp Physiol, 293(4):R1538-44), suggesting heparan sulfate as a potential therapeutic target for Pep19-4LF. Indeed, using isothermal titration calorimetry, we found a strong Coulomb interaction between Pep19-4LF and heparan sulfate, as indicated by strong exothermic reaction running into a saturation characteristic (FIG. 10C). To investigate whether Pep19-4LF inhibits the release of TNFα caused by heparan sulfate in human cells, we exposed human MNCs to heparan sulfate in the presence or absence of Pep19-4LF. Most notably, Pep19-4LF attenuated the release of TNFα caused by heparan sulfate in these cells in a dose-related fashion (FIG. 10A) indicating that Pep19-4LF is able to prevent the activation of human cells by the DAMP heparan sulfate. Thus, these findings indicate, that the organ protective and anti-inflammatory effects of Pep19-4LF in HS are, at least partly, associated with its interaction with relevant DAMPs, such as heparan sulfate.

In conclusion, we report here for the first time that trauma and trauma-haemorrhage result in a significant release of the host-defence/antimicrobial peptide LL-37. As the systemic administration of higher doses of LL-37 leads to adverse effects, we have investigated the use of synthetized, small host-defence/antimicrobial peptides, Pep19-4LF and Pep19-2.5. Like LL-37, Pep19-4LF and Pep19-2.5 neutralize the effects of LPS and lipoproteins (Tejada G Md et al (2015) Sci Rep, 5:14292). LL-37 also interacts with and attenuates the effects of several DAMPs (Martin L et al (2015) Front Immunol, 6:404; Hu Z et al (2014) PLoS One, 9(1):e85765). We report here that Pep19-4LF abolishes the release of TNFα caused by heparan sulfate in human mononuclear cells. In addition, Pep19-4LF as well as Pep19-2.5 attenuate the organ injury/dysfunction caused by severe hemorrhage and resuscitation in the anesthetized rat. This protective effect of Pep19-4LF was associated with activation of the Akt/eNOS-survival pathway (kidney and liver), which increases the resistance of these organs to injury. In addition, Pep19-4LF also attenuates the activation of NF-iB in these organs, resulting in the reduced formation of the pro-inflammatory cytokines IL-6 and MCP-1. Thus, Pep19-4LF as well as Pep19-2.5 are useful in reducing the organ injury/dysfunction and inflammation caused by severe hemorrhage and resuscitations in patients with trauma.

1. A method of preventing or treating hemorrhagic shock (HS), or ameliorating symptoms associated with HS in an individual, wherein the method comprises administering to the individual an effective amount of a peptide or a variant thereof, wherein the peptide or variant thereof is linear and non-cyclic, and comprises 17 to 21 amino acids, and wherein the amino acids in positions (1) to (21), counted from the N-terminus, are as follows:
(1) G, S or lacking;
(2) C or lacking;
(3) K or R;
(4) K or R;
(5) Y or F;
(6) K or R;
(7) K or R;
(8) F, W or L;
(9) K, R or lacking;
(10) W, L or F;
(11) K or R;
(12) F, Y or C;
(13) K or R;
(14) G or Q;
(15) K or R;
(16) F, L or W;
(17) F or W;
(18) F, L or W;
(19) W or F;
(20) C or lacking;
(21) G or lacking;
wherein when position (9) is lacking, positions (1) and (21) cannot be lacking, and wherein the variant has up to one, two or three substitutions.

2. The method according to claim 1, wherein the peptide or variant thereof comprises or has 7 to 9 positively charged amino acids and 7, 8 or 9 hydrophobic amino acids.

3. The method according to claim 1, wherein the peptide or variant thereof comprises or consists of at least one G amino acid residue, no more than six K amino acid residues, no more than four F amino acid residues, and/or no more than three L amino acid residues.

4. The method according to claim 1, wherein the peptide comprises or consists of an amino acid sequence of any one of:

| | |
|---|---|
| KKFRRLKWKYKGKFWFW; | (SEQ ID NO: 1) |
| KKYRRFRWKFKGKFWFW; | (SEQ ID NO: 2) |
| RRYKKFKWRYRGRFWFW; | (SEQ ID NO: 3) |
| GRRYRRFWKFRGKWFFWG; | (SEQ ID NO: 4) |
| GKKYRRFRWKFKGKWFWFG; | (SEQ ID NO: 5) |
| GRRYRRFRWRFRGRFWFWG; | (SEQ ID NO: 6) |
| GRRYKKFRWKFKGRWFWFG; | (SEQ ID NO: 7) |
| GRRYRRFRWRFRGRFWFWG; | (SEQ ID NO: 8) |
| GKKYRRFRWKFKGKLFLFG; | (SEQ ID NO: 9) |
| GCKKYRRFRWKFKGKFWFWG; | (SEQ ID NO: 10) |
| GCKKFRRFKLKCKQKLWLWCG; | (SEQ ID NO: 11) |
| GCKKFRRLKWKYKGKFWFWCG; | (SEQ ID NO: 12) |
| GCRRYRRFRWKFRGRFWFWCG; | (SEQ ID NO: 13) |
| GCRRYKKFKWRYRGRFWFWCG; | (SEQ ID NO: 14) |
| GCRRFRRFRWRYRGRFWFWCG. | (SEQ ID NO: 15) |

5. The method according to claim 1, wherein the amino acids in positions (1) to (21), counted from the N-terminus, are as follows:
(1) G;
(2) lacking;
(3) K or R;
(4) K or R;
(5) Y or F;
(6) K or R;
(7) K or R;
(8) F, W or L;
(9) K or R;
(10) W, L or F;
(11) K or R;
(12) F, Y or C;
(13) K or R;
(14) G or Q;
(15) K or R;
(16) L;
(17) F;
(18) L;
(19) F;
(20) lacking;
(21) G.

6. The method according to claim 5, wherein the peptide comprises or consists of GKKYRRFRWKFKGKLFLFG (SEQ ID NO: 9).

7. The method according to claim 6, wherein the peptide is in D-configuration.

8. The method according to claim 5, wherein the amino acid sequence of the variant has up to one, two or three substitutions, wherein position (1) is G; (2) is lacking; (16) is L; (17) is F; (18) is L; (19) is F; (20) is lacking; and (21) is G.

9. The method according to claim 1, wherein the amino acids in positions (1) to (21), counted from the N-terminus, are as follows:
(1) G;
(2) C;
(3) K or R;
(4) K or R;
(5) Y or F;
(6) K or R;
(7) K or R;
(8) F, W or L;
(9) K or R;
(10) W, L or F;
(11) K or R;
(12) F, Y or C;
(13) K or R;
(14) G or Q;
(15) K or R;
(16) F;
(17) W;
(18) F;
(19) W;
(20) lacking;
(21) G.

10. The method according to claim 9, wherein the peptide comprises or consists of GCKKYRRFRWKFKGKFWFWG (SEQ ID NO: 10).

11. The method according to claim 10, wherein the peptide is in D-configuration.

12. The method according to claim 9, wherein the amino acid sequence of the variant has up to one, two or three substitutions, wherein position (1) is G; (2) is C; (16) is F; (17) is W; (18) is F; (19) is W; (20) is lacking; and (21) is G.

13. The method according to claim 1, wherein the peptide is fused to a tag, signal peptide or an antigenic determinant.

14. The method according to claim 13, wherein the peptide is fused via a linker.

15. The method according to claim 1, wherein administration of the peptide attenuates HS-associated organ injury or dysfunction.

16. The method according to claim 1, wherein administration of the peptide attenuates the decline in blood pressure.

17. The method according to claim 1, wherein administration of the peptide attenuates lung inflammation.

18. The method according to claim 1, wherein the peptide binds to heparan sulfate.

19. The method according to claim 1, wherein the peptide does not show haemolytic activity.

20. The method according to claim 1, wherein the HS is trauma-associated HS, optionally wherein the plasma levels of LL-37 in the individual is elevated.

21. The method according to claim 1, wherein the peptide is administered by intravenous injection, intramuscular injection or intraosseous injection.

22. The method according to claim 1, wherein the effective amount of peptide is from about 50 μg/kg per hour to 350 μg/kg per hour.

23. The method of claim 22, wherein the peptide is administered continuously for 4 hours.

24. The method according to claim 1, wherein the method further comprises administering another therapy or agent, wherein said therapy or agent prevents or treats the HS, or ameliorates the symptoms of HS.

25. The method according to claim 24, wherein the therapy is blood transfusion, wherein the agent is intravenous crystalloids, colloidal solutions, hypertonic saline, dopamine, dobutamine, adrenaline, noradrenaline, artesunate, antibiotics, steroids, prothrombin complex concentrate (factor IX complex) and/or tranexamic acid.

26. The method according to claim 25, wherein the blood transfusion is blood plasma transfusion, platelet transfusion or red blood cell transfusion, and/or wherein the colloidal solution is albumin or hetastarch.
